(19) United States
(12) Patent Application Publication
(10) Pub. No.: US 2013/0267039 A1
(43) Pub. Date: Oct. 10, 2013

(54) Single Molecule Assays

(71) Applicant: SINGULEX, INC., Alameda, CA (US)

(72) Inventors: John Todd, Lafayette, CA (US); Quynhanh Lu, Mountain View, CA (US); Robert Freese, Waterloo, IA (US)

(73) Assignee: SINGULEX, INC., Alameda, CA (US)

(21) Appl. No.: 13/791,969

(22) Filed: Mar. 9, 2013

Related U.S. Application Data
(63) Continuation of application No. 12/562,879, filed on Sep. 18, 2009.
(60) Provisional application No. 61/098,712, filed on Sep. 19, 2008.

(51) Int. Cl. G01N 33/543; G01N 33/74; G01N 33/68

(52) U.S. Cl. CPC G01N 33/54333; G01N 33/6869; G01N 33/74
USPC 436/501

(57) ABSTRACT

The present invention provides single molecule analyses of species of use in analytical, diagnostic or prognostic assays. In exemplary embodiments, the assays utilize samples prepared by novel methods, affording assays of unexpected sensitivity and robustness. The method is described in a non-limiting manner by reference to cytokine assays.

This application is a continuation of U.S. Non-provisional application Ser. No. 12/562,879, filed Sep. 18, 2009, and claims the benefit of U.S. Provisional Pat. App. No. 61/098,712, filed Sep. 19, 2008 which is incorporated by reference in its entirety for all purposes.

### FIELD OF THE INVENTION

The present invention is directed to assays detecting one or more discrete single molecule of an analyte.

### BACKGROUND OF THE INVENTION

Advances in biomedical research, medical diagnosis, prognosis, monitoring and treatment selection, bioterrorism detection, and other fields involving the analysis of multiple samples of low volume and concentration of analytes have led to development of sample analysis systems capable of sensitively detecting particles in a sample at ever-decreasing concentrations. U.S. Pat. Nos. 4,793,705 and 5,209,834 describe previous systems in which extremely sensitive detection has been achieved. The present invention provides further development in this field, particularly in the field of cytokine detection, quantification and characterization.

Traditional cytokine assays involve measurement of cytokines in serum or plasma, but various cytokines have been detected in other biological fluids. For example, Kimball, J. Immunol. 133:256-260 (1984) reported IL-1 bioactivity in human urine. Tamatani et al., Immunology 65:337-342 (1988) disclosed the presence of IL-1α and IL-1β in human amniotic fluid, using chromatographic and bioassay methods. The same group used enzyme immunoassays to measure IL-1α and IL-1β in human amniotic fluid (Tsunoda et. al., Lymphokine Res. 7:333, 1988). Wilmott et al. Lymphokine Res. 7:334 (1988) measured IL-1β and IL-1 bioactivity in human bronchoalveolar lavage fluid in cystic fibrosis compared to other diseases. Khan et al., Mol. Cell. Endocrinol., 58:221-230 (1988) reported that high levels of IL-1-like bioactivity could be demonstrated in human ovarian follicular fluid. Lymphotoxins have been reported in blister fluid of pemphigoid patients (Jeffes et al., J. Clin. Immunol. 4:31-35, 1984). IL-1 has also been reported in human sweat (Didierjean et al., Cytokine 2:438-446, 1990). IL-1 has been reported to be found in the cerebrospinal fluid (CSF) of cats (Coceani et al., Brain Res., 446:245-250, 1988) and humans (see, for example, Peter et al., Neurology, 41:121-123, 1991). A factor with IL-1-like bioactivity was detected in the gingival fluid of clinically normal humans (Oppenheim et al., Transplant. Proc. 14:553-555, 1982), the activity being higher in inflamed than non-inflamed gingival regions.

Binding assays for measuring cytokine levels may use solid phase or homogenous formats. Suitable assay methods include sandwich or competitive binding assays. Examples of sandwich immunoassays are described in U.S. Pat. No. 4,168,146 to Grubb et al. and U.S. Pat. No. 4,366,241 to Tom et al. Examples of competitive immunoassays include those disclosed in U.S. Pat. No. 4,235,601 to Deutsch et al., U.S. Pat. No. 4,442,204 to Liotta, and U.S. Pat. No. 5,208,535 to Buechler et al.

Multiple cytokines may be measured using a multiplexed assay format, e.g., multiplexing through the use of binding reagent arrays, multiplexing using spectral discrimination of labels, multiplexing by flow cytometric analysis of binding assays carried out on particles (e.g., using the Luminex system). Another approach involves the use of binding reagents coated on beads that can be individually identified and interrogated. International Patent publication WO9926067A1 (Watkins et al.) describes the use of magnetic particles that vary in size to assay multiple analytes; particles belonging to different distinct size ranges are used to assay different analytes. The particles are designed to be distinguished and individually interrogated by flow cytometry. Vignali has described a multiplex binding assay in which 64 different bead sets of microparticles are employed, each having a uniform and distinct proportion of two dyes (Vignali, D. A. A., “Multiplexed Particle-Based Flow Cytometric Assays,” J. Immunol. Meth. (2000) 243:243-255). A similar approach involving a set of 15 different beads of differing size and fluorescence has been disclosed as useful for simultaneous typing of multiple pneumococcal serotypes (Park, M. K. et al., “A Latex Bead-Based Flow Cytometric Immunoassay Capable Of Simultaneous Typing Of Multiple Pneumococcal Serotypes (Multibead Assay),” Clin Diagn Lab Immunol. (2000) 7:486-9). Bishop, J. E. et al. have described a multiplex sandwich assay for simultaneous quantification of six human cytokines (Bishop, J. E. et al., “Simultaneous Quantification of Six Human Cytokines in a Single Sample Using Microparticle-based Flow Cytometric Technology,” Clin Chem. (1999) 45:1693-1694).

Other methods are used to quantify specific cytokine expression including methods that measure cytokine mRNA or cytokine polypeptide. For example, PCR™, competitive PCR™, PCR-ELISA, Microarrays, gene expression bead-based assays, and in situ hybridization techniques can be used to measure cytokine mRNA, and immunohistochemistry can be used to measure cytokine protein levels. Published US Patent Application No. 20060205012 discloses assay methods for measuring the levels of one or more cytokines in a sample.

A need currently exists for more sensitive methodologies for measuring low concentrations of analytes of diagnostic or prognostic value, e.g., cytokines. Furthermore, more effective and efficient methods, compounds and systems for single molecule detection of analytes would represent a significant advance in diagnosis and prognosis in various contexts. These and further needs are provided by the present invention.

### SUMMARY OF THE INVENTION

The present invention provides a method of performing assays detecting a single molecule of an analyte. The method provides detection of species of analytical, diagnostic, and prognostic interest at lower detection threshold levels than currently available methodologies. Moreover, various embodiments of the invention provide a single molecule assay having a dynamic range broader than currently available methods. The significance of the methods of the invention is further augmented by the powerful clinical need for rapid, inexpensive single- and multi-biomarker diagnostic assays requiring high sensitivity and a robust dynamic range of detection.

The methods of the present invention are distinct from microplate-based assays. Microplate-based assays do not readily allow the possibility removing substantially all unbound detection species from the complex formed between the detection species and the analyte. In various embodiments, the invention provides a method that includes separating the unbound detectable species from the complex by transferring the capture antibody-analyte-detection antibody sandwich complex to a vessel free of the detection antibody prior to eluting the detection antibody from the sandwich complex. Assay mixtures in which non-specifically bound detection antibody is present and is eluted into the detection mixture in tandem with the specifically bound detection antibody of the sandwich complex produce inaccurately high signal.

In a first aspect, the invention provides a method of performing an assay detecting a single molecule of a detectable species correlating to a single molecule of an analyte to which the detectable species specifically binds. The method includes separating essentially all unbound detectable species from the capture antibody-analyte-detection antibody sandwich complex prior to detecting the detectable species. Thus, in an exemplary embodiment, the invention provides a method including, in a first vessel, forming a complex between the analyte and a capture species specifically binding the analyte. The capture species is immobilized on a magnetic particle. Following capture of the analyte and still in the first vessel, the complex is contacted with the detectable species which specifically binds to the analyte thereby forming an immobilized complex of the detectable species. The immobilized complex is then removed from the first vessel and transferred to a second vessel, which is free from the detectable species. Once in the second vessel, the detectable species is eluted from the immobilized complex. Using an appropriate device for single molecule detection, a single molecule of the detectable species is detected.

In various embodiments, the single molecule of the detectable species is one of a plurality of molecules of detectable species having essentially the same structure. In various other embodiments, there are two or more populations of detectable species, each population including detectable species of different structure. In an exemplary embodiment, the capture species is an antibody. In various embodiments, the detectable species is a labeled antibody, e.g., an antibody labeled with a fluorophore.

In an exemplary embodiment, the invention provides a method as set forth above in which the single molecule of a detectable species correlates to a single molecule of an analyte which is a member selected from a cytokine and a growth factor, to which the detectable species specifically binds. In various embodiments, the assay is capable of detecting the detectable species in an amount of less than or equal to about 5 pg/mL. In certain embodiments, the assay detects a single molecule of the detectable species in an amount of less than or equal to about 2 pg/mL.

The method of the invention also provides assays with a robust dynamic range. For example, in various embodiments, the invention provides assays having a dynamic range of at least about 2 log, preferably at least 3 log and more preferably, at least about 4 log.

In various embodiments, the invention provides a method of determining a diagnosis, a prognosis, a state of treatment, and/or a method of treatment based on a result of a single molecule assay. The method is substantially as set forth above, with the additional elements of quantifying a plurality of single molecules of the detectable species on a single molecule by single molecule basis (as distinct from an average concentration) and correlating the amount derived from this quantification with the diagnosis, prognosis, state of treatment or method of treatment.

The invention also includes reagents and kits for carrying out the methods of the invention. In one embodiment, a kit comprises antibodies against the analyte (e.g., cytokines) being measured in a method of the invention. The kit may further comprise assay diluents, standards, controls and/or detectable labels. The assay diluents, standards and/or controls may be optimized for a particular sample matrix. For example, for measurements in blood, serum or plasma samples, the diluents, standards and controls may include i) human blood, serum or plasma; ii) animal blood, serum or plasma or iii) artificial blood, serum or plasma substitutes.

A variety of cytokines are potentially useful as diagnostic marker(s) for performing the inventive methods for the diagnosis and/or monitoring of various diseases and for screening drugs or drug candidates for efficacy in treating disease. Furthermore, as described in detail below, certain embodiments of the invention provide methods for determining the efficacy of particular candidate cytokine(s) for acting as marker(s) in these and other analyses. Using the methods of the present invention, one of ordinary skill in the art will be able to determine, without undue experimentation, the ability of one or more selected cytokines or other markers, including cytokines and other markers not specifically listed herein and indeed not yet discovered, to be useful as markers whose measured levels/profiles may be employed in performing the various diagnostic and screening methods of the invention.

Further objects, advantages and aspects of the present invention will be apparent from the detailed description that follows.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore non-limiting, embodiments illustrated in the drawings, wherein like reference numerals (if they occur in more than one view) designate the same elements. The invention may be better understood by reference to one or more of these drawings in combination with the description presented herein. It should be noted that the features illustrated in the drawings are not necessarily drawn to scale.

FIG. 1 is a schematic representation of an exemplary optical system of use in the methods of the invention.

FIG. 2 is a plot showing the accuracy of curve fit; back interpolation of cTNI standard curves generated over 8 consecutive assay runs (panel A, full range and panel B low end range of quantification).

FIG. 3 is a frequency distribution of cTnI in lithium heparin plasma specimens obtained from 100 different blood donors.

FIG. 4A is a frequency distribution of IL-2 in plasma obtained from healthy human volunteers.

FIG. 4B is a frequency distribution of IL-5 in plasma obtained from healthy human volunteers.

FIG. 4C is a frequency distribution of IL-7 in plasma obtained from healthy human volunteers.

FIG. 4D is a frequency distribution of IL-21 in plasma obtained from healthy human volunteers.

FIG. 5 is a table of concentrations of various interleukins (IL-2, IL-5, IL-7 and IL-1b) in samples from healthy volunteers measured using the method of the invention.

FIG. 5B is a table of concentrations of various interleukins (IFN-g, IL-21, IL-6 and IL-17A) in samples from healthy volunteers measured using the method of the invention.

FIG. 5C is a table of concentrations of various interleukins (TNF-a, IL-4, IL-1a and GMCSF) in samples from healthy volunteers measured using the method of the invention.

FIG. 5D is a table of concentrations of various interleukins (IL-12, G-CSF, IL-22 and IL-10) in samples from healthy volunteers measured using the method of the invention.

FIG. 5E is a table of concentrations of various interleukins (MIP-1α, IL-15, IL-22 and IL-10) in samples from healthy volunteers measured using the method of the invention.

FIG. 6 is a table showing the ratios of various interleukin concentrations from FIG. 5.

### DETAILED DESCRIPTION OF THE INVENTION

### Introduction

Disclosed herein are examples of inventive methods for detecting single molecules of species of analytic, clinical, prognostic and/or diagnostic interest in samples. In various embodiments, the samples are taken from a subject. The assays may comprise measuring analytes in biological samples, for example measuring disease markers, markers of inflammation, and/or cytokines, where the levels of the analytes are indicative of the presence or severity of a disease.

Various embodiments of the invention relate to methods for detecting and/or distinguishing various diseases, predicting the course and/or outcome of such diseases, methods of treating such diseases and agents of use in preventing or arresting the progress of such diseases. Exemplary embodiments of the invention relate to methods for monitoring the progression or treatment of a disease in a patient by administering and/or repetitively administering a diagnostic test according to the assay format of the present invention. In one example, the diagnostic methods are used to evaluate the effectiveness of a drug or drug candidate for treating a disease by measuring the effect of the drug or drug candidate on the levels of disease-specific analytes in samples from patients, animal models, tissue samples and cell cultures treated with a drug or a drug candidate.

In various embodiments, the invention provides methods for conducting diagnostic tests for the detection of diseases in which cytokines are involved or implicated. An exemplary embodiment is an assay for identifying diagnostically valuable cytokine markers of diseases. In various embodiments, the invention provides methods for determining the efficacy of particular candidate analytes, such as particular cytokine(s), for acting as diagnostic marker(s) in the inventive methods for the diagnosis and/or monitoring of a disease and for screening drugs or drug candidates for efficacy in treating a disease.

In various embodiments, only specific single molecules within a mixture are labeled. Specific labeling can be accomplished by combining the target single molecule with a labeled binding partner, where the binding partner interacts specifically with the target single molecule through complementary binding surfaces. Binding forces between the partners can be covalent interactions or non-covalent interactions such as hydrophobic, hydrophilic, ionic and hydrogen bonding, van der Waals attraction, or coordination complex formation. Examples of binding partners are agonists and antagonists for cell membrane receptors, toxins and venoms, antibodies and viral epitopes, hormones (e.g., opioid peptides, steroids, etc.) and hormone receptors, enzymes and enzyme substrates, cofactors and target sequences, drugs and drug targets, oligonucleotides and nucleic acids, proteins and monoclonal antibodies, antigen and specific antibody, polynucleotide and complementary polynucleotide, polynucleotide and polynucleotide binding protein; biotin and avidin or streptavidin, enzyme and enzyme cofactor; and lectin and specific carbohydrate. Illustrative receptors that can act as a binding partner include naturally occurring receptors, e.g., thyroxine binding globulin, lectins, various proteins found on the surface of cells (cluster of differentiation or CD single molecules), and the like.

In one embodiment, a sample is reacted with beads or microspheres, e.g., magnetic particles, that are coated with a binding partner, e.g., a capture species, that reacts with the target single molecule. The beads are separated from any non-bound components of the sample. In one embodiment, the captured analyte is contacted with a detectable species, and following removal of non-bound material, the analyte-detectable species complex is transferred to a separate vessel in which it is optionally washed and is treated with an elution mixture, which dissociates the analyte from the detectable species. Individual molecules are of the detectable species are detected by a single molecule analyzer of use in the invention.

### DEFINITIONS

As used herein, the terms “analyte” and “binding partner” refer to molecules involved in binding interactions. By way of example, analyte and binding agent may include any organic, inorganic or biological agent. Non-limiting examples of such agents include, DNA or RNA fragments (e.g., oligonucleotides), aptamers, peptides, and proteins (e.g., antibodies), antigens and small organic molecules (e.g., pharmaceutical agents, agents of war, pesticides, herbicides, agricultural fertilizers). In a particular assay, binding interactions between ligands and receptors are analyzed. In an exemplary assay, binding of the analyte and the binding partner is a step in providing a detectable species, one or more molecule of which can be detected in the assays of the invention. The detectable species is thus a proxy for the analyte: a molecule of a detectable species correlates and corresponds to a molecule of an analyte. Though the correlation is not necessarily one-to-one, it is deducible or experimentally determinable. An analyte is derived from any prokaryotic or eukaryotic individual, including an animal (e.g., a mammal, e.g., a human; bird, fish, etc.), a plant, a yeast, a bacteria, a virus, a protozoa and the like. The invention is not limited with respect to the origin of the analyte or of the sample containing the analyte.

As used herein, “sample” includes material from essentially any source of interest. Non-limiting examples of samples include those selected from the group consisting of blood, serum, plasma, bronchoalveolar lavage fluid, urine, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the analyte. In some embodiments, the sample is selected from the group consisting of blood, plasma, or serum. In some embodiments of the methods of the invention, the sample is a serum sample that has been contacted with a fluorescently-labeled antibody specific for a analyte molecule of interest; and wherein said analysis comprises detecting the presence, absence, and/or concentration of the labeled analyte molecule. In some embodiments, concentration is determined by counting and quantifying the number of analyte molecules of the detectable species in a selected volume unit. In some of these embodiments, the method further includes determining a diagnosis, prognosis, state of treatment, and/or method of treatment, based on said presence, absence, and/or concentration of the labeled analyte molecule.

“Binding partner,” as used herein refers to a species that binds to an analyte of interest in an assay of the invention. Exemplary binding partners include “capture species” and “detectable species” are used essentially interchangeably. Non-limiting examples of binding partners include a protein (e.g., antibody, receptor), a nucleic acid, a carbohydrate, an amino acid, a lipid, a toxin, a venom, a drug, a virus, a bacterium, a cell, and any combination thereof. In various embodiments when the binding partner is a detectable species, it is labeled with a detectable label. When the binding partner is a capture species, in some embodiments, it is bound to a magnetic particle through a covalent or non-covalent interaction.

“Detectable species,” as used herein, refers to a first detectable binding partner for an analyte or a detectable binding partner for a different binding partner binding with the analyte. In an exemplary embodiment, the detectable species binds specifically to the analyte or to the different binding partner for the analyte. In various embodiments, a single molecule of the detectable species is detectable using the methods of the invention. In many embodiments of the invention, basic knowledge in the art relating to immunoassays (e.g., sandwich, Elisa, etc.) is applicable and an exemplary detectable species is an antibody conjugated to a detectable label. In certain embodiments, a single molecule of a detectable species is detectable by a method of the invention. In various embodiments, the detectable species binds the analyte in a reversible manner, thus, upon a change of the ambient milieu, the analyte is released from the detectable species. In a preferred embodiment, the detectable species is a proxy for the analyte, correlating to a single molecule of the analyte in a deducible or experimentally determinable manner.

“Capture species,” refers to a binding partner for an analyte, specifically recognizing the analyte, which allows for the analyte to be separated from at least one non-analyte contaminating substance in a sample. In various embodiments, the complex formed between the analyte and the capture species is stable through one or more washing step, allowing the analyte to be separated from essentially all contaminating substances. In various embodiments, the capture species binds the analyte in a reversible manner, thus, upon a change of the ambient milieu, the analyte is released from the capture species. An exemplary capture species is an antibody, e.g., an antibody to a cytokine, growth factor or other biologically relevant analyte.

As used herein, the term “magnetic particle” refers to a magnetic bead or particle possessing a permanent or induced dipole moment. Exemplary magnetic particles of use in the invention include a reactive functionality allowing conjugation between the particle and a binding partner (“capture species”) for the analyte and the particle. Essentially any format of magnetic particle having this feature is of use in the present invention. For example, polysaccharide coated paramagnetic microspheres or nanospheres may be used to label particles. U.S. Pat. No. 4,452,773 issued to Molday, describes the preparation of magnetic iron-dextran magnetic particles and provides a summary describing the various methods of preparing particles suitable for attachment to biological materials. A description of polymeric coatings for magnetic particles used in high gradient magnetic separation methods are found in German Patent No. 3720844 and U.S. Pat. No. 5,385,707 issued to Miltenyi, both incorporated herein by reference in their entireties. Methods to prepare paramagnetic magnetic particles are described in U.S. Pat. No. 4,770,183.

Magnetic particles are functionalized with binding partner molecules attached thereto by any appropriate methodology. For example, magnetic particles can be conjugated to nucleic acids, e.g., DNA (oligonucleotides) or RNA fragments, peptides or proteins, aptamers and small organic molecules in accordance with processes known in the art, e.g., with one of several coupling reactions of the known art (G. T. Hermanson, Bioconjugate Techniques (Academic Press, 1996); L. Ilium, P. D. E. Jones, Methods in Enzymology 112, 67-84 (1985). In certain embodiments of the invention, the functionalized magnetic particles have binding partner molecules (e.g., DNA, RNA or protein) covalently bound thereto. The binding partner molecule is bound to the magnetic particle through a non-covalent (e.g., van der Waals, hydrophobic-hydrophobic, hydrophilic-hydrophilic or ionic) interaction. In an exemplary embodiment, the binding partner molecule is conjugated to the magnetic particle through the biotin streptavidin interaction. In various embodiments, the magnetic particle is functionalized with streptavidin and the binding partner is functionalized with avidin. In other embodiments, the magnetic particle is functionalized with biotin and the binding partner is conjugated to streptavidin.

Functionalization of the magnetic particle with binding partner molecules typically requires one-step or two-step reactions which may, for example, be performed in parallel using standard liquid handling robotics and a 96-well format to attach any of a number of desirable binding partners to designated magnetic particles. Samples may be drawn for QC measurements. Each batch of magnetic particles potentially provides material for multiple assays so that assay-to-assay variations are minimized Magnetic particles may be stored in a buffered bulk suspension until needed.

The exact method for attaching the analyte to the magnetic particle-immobilized capture species is not critical to the practice of the invention, and a number of alternatives are known in the art. The attachment is generally through interaction of the analyte molecule with a specific binding partner which is conjugated to the coating on the magnetic particle and provides a functional group for the interaction. Antibodies are examples of binding partners. Antibodies may be coupled to one member of a high affinity binding system, e.g., biotin, and the analyte molecule attached to the other member, e.g., avidin. Secondary antibodies recognizing species-specific epitopes of the primary antibodies, e.g., anti-mouse Ig, and anti-rat Ig, may also be used in the present invention. Indirect coupling methods allow the use of a single magnetically coupled entity, e.g., antibody, avidin, etc., with a variety of analyte molecules.

As used herein, the term “label” refers to any species that makes one or more single molecule or one or more detectable species individually detectable in a single molecule analyzer of use in the invention. Labels of use in the present invention include dye tags, charge tags, mass tags, Quantum Dots, or beads, magnetic tags, light scattering tags, polymeric dyes, and dyes attached to polymers.

Fluorescent labels, or dyes, are a non-limiting example of a type of label of use in the present invention. Examples of fluorescent labels can be found in the HANDBOOK OF FLUORESCENT PROBES AND RESEARCH PRODUCTS (R. Haugland, 9th Ed., Molecular Probes Pub. (2004)). Dyes include a very large variety of compounds that add color to materials and/or enable generation of luminescent or fluorescent light. A dye may absorb light or emit light at specific wavelengths. A dye may be intercalating, or be noncovalently or covalently bound to an analyte molecule. Dyes themselves may constitute probes detecting various structures on an analyte, e.g., minor groove structures, cruciforms, loops or other conformational elements of analyte molecules. Dyes may include BODIPY and ALEXA dyes, Cy[n] dyes, SYBR dyes, ethidium bromide and related dyes, acridine orange, dimeric cyanine dyes such as TOTO, YOYO, BOBO, TOPRO POPRO, and POPO and their derivatives, bis-benzimide, OliGreen, PicoGreen and related dyes, cyanine dyes, fluorescein, LDS 751, DAPI, AMCA, Cascade Blue, CL-NERF, Dansyl, Dialkylaminocoumarin, 4′,5′-Dichloro-2′,7′-dimethoxyfluorescein, 2′,7′-Dichlorofluorescein, DM-NERF, Eosin, Erythrosin, Fluoroscein, Hydroxycourmarin, Isosulfan blue, Lissamine rhodamine B, Malachite green, Methoxycoumarin, Naphthofluorescein, NBD, Oregon Green, PyMPO, Pyrene, Rhodamine, Rhodol Green, 2′,4′,5′,7′-Tetrabromosulfonefluorescein, Tetramethylrhodamine, Texas Red, X-rhodamine, Dyomic dye series, Atto-tec dye series, Coumarins, phycobilliproteins (phycoerythrins, phycocyanins, allophycocyanins), green, yellow, red and other fluorescent proteins, up-converting phosphors, and Quantum Dots. Those skilled in the art will recognize other dyes which may be used within the scope of the invention. This is not an exhaustive list, and acceptable dyes include all dyes now known or known in the future which could be used to allow detection of the labeled analyte molecule using a method of the invention.

In an exemplary embodiment, the detectable label is a luminescent label, or a light scattering label. In one embodiment, the detectable label is a luminescent label. Although other luminescent labels may be used without departing from the scope of the present invention, useful luminescent labels include fluorescent labels, chemiluminescent labels, and bioluminescent labels, among others. In addition, fluorescent quenching can also be monitored. Additionally, other light scattering labels may be used without departing from the scope of the present invention. Useful light scattering labels include metals, such as gold, silver, platinum, selenium and titanium oxide, among others. A detectable label may also be produced by any combination of intrinsic and extrinsic properties of the analyte molecule.

Light scattering tags which may be used in the present invention include metals such as platinum, gold, silver, selenium and titanium oxide. Those of skill in the art will recognize other microspheres or beads can also be used as light scattering tags. Other useful labels include, labels affecting the electrophoretic velocity and/or separation of target analyte molecules of identical or different sizes that cannot be separated electrophoretically. Such labels are referred to as charge/mass tags. The attachment of a charge/mass tag alters the ratio of charge to translational frictional drag of the target analyte molecules in a manner and to a degree sufficient to affect their electrophoretic mobility and separation.

In another embodiment, the label alters the charge, or the mass, or a combination of charge and mass. The charge/mass tag bound to an analyte molecule can be discriminated from the unbound analyte molecule or unbound tag by virtue of spatial differences in their behavior in an electric field or by virtue of velocity differences in their behavior in an electric field.

Methods for labeling a binding partner for an analyte molecule, converting the binding partner to a detectable species are well known by those of ordinary skill in the art. Attaching labels to analyte molecules can employ any known method including attaching directly or using binding partners. In some cases, the method of labeling is non-specific. For example, methods are known that label all nucleic acids regardless of their specific nucleotide sequence. In other cases, the labeling is specific, as in where a labeled oligonucleotide binds specifically to a target nucleic acid sequence.

In some embodiments, the methods of the invention include performing an analysis on a plurality of analyte molecules in the sample. In some of these embodiments, each detected analyte molecule of the plurality of analyte molecules comprises a label, and wherein each detected analyte molecule is distinguished from the others by a characteristic selected from the group consisting of label identity, label intensity, mobility, or a combination thereof.

The term “bin,” as used herein refers to a predetermined time during which a signal from a detectable species is detected/measured. In an exemplary embodiment, the bin is a unit of time during which photons from a luminescent label are detected/measured.

The term “event photon(s)” refers to a collection of photons detectable in a method of the invention. The event photon(s) are those photons that are located within a “bin” at a given point in time at which the photons in the bin are detected. The event photons are quantifiable as a measure of the amplitude of light within the bin.

The term “total photon(s) refers to a collection of photons detectable in a method of the invention. The total photons are measured by summing all of the photons in the bins

### THE EMBODIMENTS

In a first aspect, the invention provides a method of performing an assay detecting an analyte molecule of a detectable species correlating to a analyte molecule of an analyte to which the detectable species specifically binds. In this and other methods of the invention it is generally preferred that essentially all detectable species not specifically bound to the analyte is removed from the assay mixture before the detectable species is detected, measured and/or quantified.

In various embodiments, the method of the invention includes, in a first vessel, forming a complex between the analyte and a capture species specifically binding the analyte. The capture species is immobilized on a magnetic particle. Following capture of the analyte and still in the first vessel, the complex is contacted with the detectable species which specifically binds to the analyte thereby forming an immobilized complex of the detectable species. The immobilized complex is then removed from the first vessel and transferred to a second vessel, which is free from the detectable species. Once in the second vessel, the detectable species is eluted from the immobilized complex. Using an appropriate device for analyte molecule detection, a single molecule of the detectable species is detected.

In certain embodiments, the single molecule of the detectable species is one of a plurality of molecules of detectable species having essentially the same structure. In various other embodiments, there are two or more populations of detectable species, each population including detectable species of a distinguishable different structure.

In various embodiments, the invention provides a method of determining a diagnosis, a prognosis, a state of treatment and method of treatment based on a result of an assay of the invention. An exemplary method is includes the steps set forth above, with the additional elements of quantifying a plurality of analyte molecules of the detectable species on a single molecule by single molecule basis (as distinct from an average concentration) and correlating the amount derived from this quantification with the diagnosis, prognosis, state of treatment or method of treatment.

Methods for detecting at least one analyte molecule using a single molecule analyzer are also provided. A particular feature of a useful single molecule analyzer is the ability to detect a wide range of analyte molecules. Analyte molecules which can be detected by the analyzer include, but are not limited to, organic and inorganic molecules and biological agents, supramolecular complexes, organelles, beads, associations of molecules, associations of supramolecular complexes, and organisms. Non-limiting examples of analtyes detectable using the analyzer and methods of the present invention include: biopolymers such as proteins, e.g., cytokines, nucleic acids, carbohydrates, and small molecule chemical entities, both organic and inorganic. Examples of the latter include, but are not limited to anti-autoimmune deficiency syndrome substances, antibodies, anti-cancer substances, antibiotics, anti-viral substances, enzymes, enzyme inhibitors, neurotoxins, opioids, hypnotics, antihistamines, tranquilizers, anti-convulsants, muscle relaxants and anti-Parkinson substances, anti-spasmodic and muscle contractants, miotics and anti-cholinergics, immunosuppressants (e.g., cyclosporine) anti-glaucoma solutes, anti-parasite and/or anti-protozoal solutes, anti-hypertensives, analgesics, anti-pyretics and anti-inflammatory agents (such as Non-Steroidal Antiinflammatory Drugs), local anesthetics, ophthalmics, prostaglandins, anti-depressants, anti-psychotic substances, anti-emetics, imaging agents, specific targeting agents, neurotransmitters, proteins and cell response modifiers.

Similarly, detectable chemical entities encompass small molecules such as amino acids, nucleotides, lipids, sugars, drugs, toxins, venoms, substrates, pharmacophores, and any combination thereof. Proteins are also of interest in a wide variety of therapeutics and diagnostics, such as detecting cell populations, blood type, pathogens, immune responses to pathogens, immune complexes, saccharides, lectins, naturally occurring receptors, and the like. Other examples of detectable molecules include nanospheres, microspheres, dendrimers, chromosomes, organelles, micelles and carrier molecules.

Also detectable in the methods of the invention are single species composed of complexes of single molecules, organisms with labels bound, complexes of two or more nucleic acids, and complexes of target single molecules bound to one or more antibodies or antibody fragments. Exemplary complexes where two or more types of single molecules are detected include single molecules selected from a protein, a receptor, a DNA, a RNA, a PNA, a LNA, a carbohydrate, an organelle, a virus, cell, a bacterium, a fungus, fragments thereof, and combinations thereof. Those of skill in the art will recognize how to adapt the analyzer and related methods of the present invention, in light of the numerous examples provided herein, to detect these and other single molecules.

In one embodiment, chemical entities detected by the methods of the invention include synthetic or naturally occurring hormones, naturally occurring drugs, synthetic drugs, pollutants, allergens, affecter molecules, growth factors, chemokines, cytokines, lymphokines, amino acids, oligopeptides, chemical intermediates, nucleotides, and oligonucleotides.

Methods of the invention include detecting the presence, absence, and/or concentration of a plurality of types of single molecules that have a common association, or that provide desired information, i.e., a “panel,” in a sample. A “panel,” as used herein, encompasses a group of analyte molecules whose presence may be detected by an assay of the invention. The analyte molecules may have intrinsic characteristics that allow their detection by the system of the invention, or may require labeling in order to be detected. Thus, the methods of the invention can include contacting samples with an appropriate label or plurality of labels for the detection of the presence, absence, and/or concentration of one or more members of a panel of analyte molecules. Such panels of analyte molecules are useful in, e.g., bioterrorism sample analysis, medical examination, diagnosis, prognosis, monitoring and/or treatment selection; biomedical research, forensics, agricultural analysis, and industrial applications. For example, panels may be associated with a particular type of diagnosis, e.g., panels of infectious organisms, panels of markers for disease such as cardiovascular disease, cancer or specific types of cancer, diabetes, arthritis, Alzheimer's disease, etc., or to assess functioning of various systems, e.g., endocrine panels, panels may be associated with bioterrorism, e.g., panels of likely bioterrorism organisms or toxins; panels may be useful in medical screening, e.g., panels of proteins associated with particular genetic polymorphisms or mutations associated with specific disease or pathological conditions, or associated with normal or supranormal conditions; panels may be associated with prognosis, e.g., panels of markers associated with particular syndrome, disease or disorder (e.g., cancer) may be used to determine the recurrence and/or progression of the syndrome, disease or disorder after treatment to eradicate the syndrome, disease or disorder. Panels are also useful in screening of blood samples and may include a number of infectious agents and/or antibodies for which the blood is to be screened. Similarly, a single sample may be analyzed in the methods of the invention to detect any of a number of substances of abuse, environmental substances, or substances of veterinary importance. An advantage of the invention is that it allows one to assemble a panel of tests that may be run on an individual suspected of having a disorder, disease or syndrome to simultaneously detect a causative agent for the disorder, disease or syndrome. Other areas where panels are useful include in research.

The invention also includes reagents and kits for carrying out the methods of the invention. In one embodiment, a kit comprises antibodies against the analyte (e.g., cytokines) being measured in a method of the invention. The kit may further comprise assay diluents, standards, controls and/or detectable labels. The assay diluents, standards and/or controls may be optimized for a particular sample matrix. For example, for measurements in blood, serum or plasma samples, the diluents, standards and controls may include i) human blood, serum or plasma; ii) animal blood, serum or plasma or iii) artificial blood, serum or plasma substitutes.

As an example of analyte protein molecules detectable by the present invention, it is noted that a variety of cytokines are potentially useful as diagnostic marker(s) for performing the inventive methods for the diagnosis and/or monitoring of various diseases and for screening drugs or drug candidates for efficacy in treating disease. Indeed, as described in more detail below, certain embodiments of the invention provide methods for determining the efficacy of particular candidate cytokine(s) for acting as such diagnostic marker(s). Using the methods of the present invention, one of ordinary skill in the art will be able to determine without undue experimentation the ability of one or more selected cytokines or other markers, including cytokines and other markers not specifically listed herein and indeed not yet discovered, to be useful as markers whose measured levels/profiles may be employed in performing the various diagnostic and screening methods of the invention.

### Cytokines

In various embodiments, the analyte molecules detected using the assay methods of the present invention include inflammatory markers, such as cytokines, secreted proteins that are involved in regulation of immune response. Cytokines include the interleukins (ILs), interferons (IFNs), chemokines, tumor necrosis factors (TNFs), and a variety of colony stimulating factors (CSFs). For both research and diagnostics, cytokines are useful as markers of a number of conditions, diseases, pathologies, and the like, and may be included in several different panels. There are currently over 100 cytokines/chemokines whose coordinate or discordant regulation is of clinical interest. Exemplary cytokines that are presently used in marker panels and that may be used in panels used in methods and compositions of the invention include, but are not limited to, BDNF, CREB pS133, CREB Total, DR-5, EGF, ENA-78, Eotaxin, Fatty Acid Binding Protein, FGF-basic, G-CSF, GCP-2, GM-CSF, GRO-KC, HGF, ICAM-1, IFN-alpha, IFN-gamma, IL-10, IL-11, IL-12, IL-12 p40, IL-12 p40/p70, IL-12 p70, IL-13, IL-15, IL-16, IL-17, IL-18, IL-1alpha, IL-1beta, IL-1ra, IL-1ra/IL-IF3, IL-2, IL-3, IL-4, IL-5, IL-6, IL-7, IL-8, IL-9, IP-10, JE/MCP-1, KC, KC/GROa, LIF, Lymphotacin, M-CSF, MCP-1, MCP-1 (MCAF), MCP-3, MCP-5, MDC, MIG, MIP-1 alpha, MIP-1 beta, MIP-1 gamma, MIP-2, MIP-3 beta, OSM, PDGF-BB, RANTES, Rb (pT821), Rb (total), Rb pSpT249/252, Tau (pS214), Tau (pS396), Tau (total), Tissue Factor, TNF-alpha, TNF-beta, TNF-RI, TNF-RII, VCAM-1, VEGF. The term cytokines, as used herein, also includes soluble cytokine receptors.

As those of skill will appreciate, cytokines are a representative example of useful markers detectable by the methods of the invention. The focus on cytokines is for clarity of illustration and the methods set forth herein referencing cytokines are equally apt for the detection of analyte molecules of other markers.

In various embodiments, in order to correlate a specific disease process with changes in cytokine levels, a promising approach analyzes each sample for multiple cytokines using a method of the invention.

According to various embodiments of the invention, the levels of cytokine or other disease marker candidates are measured in the samples collected from individuals clinically diagnosed with or suspected to have a disease (e.g., using conventional diagnostic methods, such as doctor's interview, endoscopies, imaging and/or biopsy) and from healthy individuals. Within non-limiting examples of this invention, specific cytokines valuable as a marker for distinguishing between normal and diseased patients could be identified using visual inspection of the data, for example, data plotted on a one-dimensional or multi-dimensional graph, or by using methods of statistical analysis, such as a statistically weighted difference between control individuals and diseased patients and/or Receiver Operating Characteristic (ROC) curve analysis. Additional modes of data analysis of use in this and other embodiments of the invention are set forth hereinbelow.

In some embodiments of the invention, determining the presence or extent of disease may comprise comparing the levels of one or more cytokines to cytokine profiles indicative of the absence, presence or extent of the disease. In one example, the levels of one or more cytokines in blood, serum and plasma samples are compared to cytokine profiles indicative of the presence or extent of the disease. In an exemplary embodiment, the step of comparing may comprise comparing cytokine levels to detection cut-off values, comparing ratios of cytokine levels to detection cut-off ratio values; comparing levels of two cytokines to detection cut-off lines in correlation plots of the two analytes, comparing levels of multiple cytokines to detection cut-off curves or surfaces in multi-analyte correlation plots and/or comparing levels of multiple cytokines to detection zones (e.g., detection areas or detection volumes) in multi-analyte correlation plots.

One embodiment of the invention includes a method for diagnosing a disease. The method includes measuring the level of a cytokine in a sample using single molecule detection as set forth herein; and diagnosing from the measured level the presence or absence in the subject of the disease.

In various embodiments of the invention, there is provided a method for detecting a disease comprising: measuring the level of cytokine in a sample, for example, a sample obtained from a patient suspected of having the disease; and diagnosing from the measured level the presence or absence in said patient of the disease. In one embodiment, the sample is a blood, serum or plasma sample.

In some embodiments of the invention, the method involves determining from measured cytokine levels if the patient has an inflammatory disease and/or determining from measured cytokine levels, the level of inflammation due to an inflammatory disease and/or obtaining and measuring samples at different times to monitor the progression of an inflammatory disease or the effectiveness of treatments for such disease. In one embodiment, the method includes measuring the level of cytokine.

In certain embodiments, the invention provides a method including measuring a plurality of cytokines and may also include comparing the levels of these cytokines to cytokine profiles determined to be indicative of the disease. A variety of samples may be analyzed. In certain embodiments, the samples may be obtained by a non-surgically invasive procedure from a human patient and may, for example, include blood, serum, plasma, fecal, or urine samples.

A particular level of a particular cytokine can be determined to be high or low based on the levels measured from various populations. An exemplary population includes, without limitation, populations of patients with ankylosing spondylitis, patients with extra-articular involvement, patients with axial joint destruction, and healthy individuals. Other cytokines identified with particular syndromes, diseases or disorders are known to those of skill in the art and can be utilized in the methods disclosed and exemplified herein.

It is recognized in the art that patients presenting similar disease symptoms can have different levels of particular cytokines within their serum, plasma, synovial fluid, tissue, cerebrospinal fluid, or other body fluid samples. Thus, determining the peripheral blood, serum, plasma, synovial fluid, tissue, cerebrospinal fluid, or other body fluid cytokine profile can be used to determine the proper treatment protocol for each individual patient. For example, two patients having similar symptoms may have different levels of IL-10 within their serum. The patient with low levels may benefit from a treatment of IL-10 while the patient with high levels of IL-10 may benefit from treatment with IL-10 inhibitors such as anti-IL-10 antibody drugs, a class of pharmaceuticals called biological drugs. Thus, determining the cytokine profile from a patient can help provide doctors and patients with information that can be used to determine adequate treatments and measure an individual patient's response to treatment.

One embodiment of the invention includes a method comprising: measuring a level of a first cytokine, for example, measuring in a sample obtained from a patient that has or is suspected to have an inflammatory disease; measuring the level of one or more additional cytokines, wherein the one or more additional cytokines differ from the first cytokine; and determining from measured levels the extent of inflammation from the disease.

Exemplary methods for distinguishing types of analyte cytokine molecules from each other are described herein. In particular, methods for detecting single molecules of a detectable species correlating to a single molecule of an analyte may use a combination label signal intensity (e.g., different numbers of label on different single molecules), label identity (e.g., different labels on different single molecules), and label mobility (e.g., different mobility for different single molecules) when motive force is electrokinetic), or combinations thereof.

Certain embodiments of the methods of the invention may further distinguish a first disease from a second disease on the basis of the measured level of a selected cytokine. For example, an exemplary method of distinguishing a first disease from a second disease includes comparing measured cytokine level to a discrimination cut-off value, wherein the measured level below the discrimination cut-off value is considered indicative of Crohn's disease and above the discrimination cut-off value is considered indicative of ulcerative colitis.

In an exemplary embodiment, determining from the first cytokine level and from one or more additional cytokine levels if the patient has a disease includes comparing one or more cytokine level to a cytokine profile determined to be indicative of the disease. The step of comparing may comprise comparing cytokine levels to detection cut-off values, comparing ratios of levels to detection cut-off ratio values and/or comparing levels to detection cut-off lines, curves or surfaces in multi-analyte correlation plots.

The step of comparing may comprise comparing levels to discrimination cut-off values, comparing ratios of levels to discrimination cut-off ratio values, and/or comparing levels to discrimination cut-off lines

In one embodiment, a first disease is distinguished from a second disease by comparing the cytokine level to a cytokine discrimination cut-off value, wherein a cytokine level below the cytokine discrimination cut-off value is considered indicative of the first disease and above the cytokine discrimination cut-off value is considered indicative of the second disease. In yet another embodiment, ulcerative colitis is distinguished from Crohn's disease by comparing the cytokine level to a cytokine discrimination cut-off line, wherein cytokine level below the cytokine discrimination cut-off line is considered indicative of Crohn's disease and above the cytokine discrimination cut-off line is considered indicative of ulcerative colitis.

In yet another embodiment, a first disease is distinguished from a second disease by comparing a measured cytokine level to a cytokine profile defined as areas situated between a first detection cut-off line and a second discrimination cut-off line on a correlation plot.

In yet another embodiment, there is provided a method for determining if a patient with an inflammatory or autoimmune disease state is predisposed to develop severe inflammatory or autoimmune disease state, comprising (a) obtaining a patient sample; (b) measuring the level of a one or a plurality of cytokines within the patient sample according to the method of the invention; (c) comparing the measured cytokine levels with predefined levels of the cytokines found in patients developing or having severe an inflammatory or autoimmune disease state; and (d) determining if said patient is predisposed to develop severe an inflammatory or autoimmune disease state.

In one embodiment, ulcerative colitis is distinguished from Crohn's disease by comparing two or more cytokines measured in a patient to a profile of these two or more cytokines, e.g., values, ratios, lines or zones on the correlation plot, indicative of a patient having ulcerative colitis, a patient having Crohn's disease and a healthy individual.

In one embodiment, a first cytokine level above a cytokine detection cut-off value and a level of an additional cytokine below a cytokine detection cut-off value are considered indicative of a disease. In another embodiment, a ratio of the first cytokine level to one additional cytokine level above a detection cut-off ratio value is considered indicative of a disease. In yet another embodiment, a cytokine level above a cytokine detection cut-off line is considered indicative of a disease.

In various other embodiments of the invention there is provided a method for evaluation of the effectiveness of a drug or drug candidate for treating a disease. For example, the invention includes a method for evaluating the effectiveness of a drug and/or drug candidate comprising: exposing a human or non-human animal with a disease and/or a model system, for example, a tissue, cell culture or a biochemical system, to the drug or drug candidate; measuring the levels of cytokine in a sample obtained from the human or non-human animal or a model system; and determining from the cytokine level measured by the method of the invention the effectiveness of the drug or drug candidate.

In another example, the invention includes a method for evaluating the effectiveness of a drug or drug candidate comprising: exposing a human or non-human animal with a disease and/or a model system, for example, a tissue, cell culture or a biochemical system, to the drug or drug candidate; measuring the level of a first cytokine in a sample obtained from the human or non-human animal or a model system; measuring the level of one or more additional cytokines in the same sample or a different sample obtained from the same human or non-human animal or a model system, wherein the one or more additional cytokines differ from the first cytokine; and determining from measured levels the effectiveness of the drug or drug candidate.

The method may also include comparing one or more cytokine levels to the levels in a control human or non-human animal that was not treated with the drug or drug candidate. The human or non-human animal in these drug evaluation methods may be replaced with an in vitro disease model system, for example, tissue, cell culture or biochemical systems that model the behavior of the disease.

Also provided is a method for assessing treatment for an inflammatory or autoimmune disease state comprising (a) subjecting an inflammatory or autoimmune disease state patient to a treatment; (b) obtaining a sample from said patient; (c) measuring the level of a plurality of cytokines within the patient sample; (d) comparing the level of a plurality of cytokines with predefined cytokine levels; and (e) determining if the treatment is efficacious.

The method may further comprise making a decision regarding modifying the therapeutic regimen based on the determination of efficacy. In various embodiments, the patient sample includes peripheral blood, serum, plasma, cerebrospinal fluid, tissue sample, skin, or other body fluid sample. The predefined levels may comprise information about a median level of the cytokine found in the patient sample. The predefined levels may comprise pretreatment levels of one or more cytokines, levels of one or more cytokines observed in healthy subjects and/or a patient with the disease. In an exemplary embodiment, the patient sample is from a patient having extra-articular involvement and/or having major joint destruction.

According to one embodiment of the invention, diagnostically valuable cytokines may be selected from a group set forth hereinbelow. Moreover, according to this embodiment, the diagnosis, prognosis and course of treatment of the various indications associated with these cytokines may be determined using the methods of the invention.

Interleukin-1α (IL-1α) is expressed in large amounts by human keratinocytes. IL-1-α is also produced also by activated macrophages from different sources (alveolar macrophages, Kupffer cells, adherent spleen and peritoneal macrophages) and also by peripheral neutrophil granulocytes, endothelial cells, fibroblasts, smooth muscle cells, keratinocytes, Langerhans cells of the skin, osteoclasts, astrocytes, epithelial cells of the thymus and the cornea, T-cells, and B-cells.

The synthesis of IL-1 can be induced by other cytokines including TNF-α, IFN-α. IFN-γ, and IFN-β and also by bacterial endotoxins, viruses, mitogens, and antigens. In human skin fibroblasts, IL-1α and TNF-α induce the synthesis of IL-1β. Human mononuclear cells are very sensitive to bacterial endotoxins and synthesize IL-1 in response to picogram/mL amounts of endotoxins. In human monocytes bacterial lipopolysaccharides induce approximately tenfold more mRNA and the respective proteins for IL-1β than for IL-1α.

The main biological activity of IL-1 is the stimulation of T-helper cells, which are induced to secrete IL-2 and to express IL-2 receptors. Virus-infected macrophages produce large amounts of an IL-1 inhibitor (IL-1ra) that may support opportunistic infections and transformation of cells in patients with T-cell maturation defects.

IL-1 stimulates the proliferation and activation of NK-cells and fibroblasts, thymocytes, glioblastoma cells. It also promotes the proliferation of astroglia and microglia and may be involved in pathological processes such as astrogliosis and demyelination. IL-1 appears to be an autocrine growth modulator for human gastric and thyroid carcinoma cells. IL-1 also has antiproliferative and cytocidal activities on certain tumor cell types. IL-1 plays an important role in pathological processes such as venous thrombosis, arteriosclerosis, vasculitis, and disseminated intravasal coagulation.] IL-1 markedly enhances the metabolism of arachidonic acid (in particular of prostacyclin and PGE2) in inflammatory cells such as fibroblasts, synovial cells, chondrocytes, endothelial cells, hepatocytes, and osteoclasts. IL-1 activates osteoclasts and therefore suppresses the formation of new bone. Low concentrations of IL-1, however, promote new bone growth. IL-1 inhibits the enzyme lipoprotein lipase in adipocytes. In vascular smooth muscle cells and skin fibroblasts IL-1 induces the synthesis of bFGF, which is a mitogen for these cells. Like Interleukin-2 (IL-2), IL-1 also modulates the electrophysiological behavior of neurons. IL-1 also directly affects the central nervous system.

IL-2 is increasingly used to treat patients with cancers refractory to conventional treatment (Waldmann et al., Annals of the New York Academy of Sciences 685: 603-10, 1993). Objective and long-lived clinical responses have been documented also in a proportion of patients with melanoma or acute myeloid leukemia (Broom et al., British Journal of Cancer. 66: 1185-7, 1992).

IL-4 is thought to be an autocrine growth modulator for Hodgkin's lymphomas (Okabe et al., Lymphoma 8: 57-63, 1992).

IL-4 may be of clinical importance in the treatment of inflammatory diseases and autoimmune diseases since it inhibits the production of inflammatory cytokines such as IL-1, IL-6, and TNF-α by monocytes and T-cells. IL4 inhibits the growth of colon and mammary carcinomas (Toi et al., Cancer Research 52: 275-9, 1992). It has been shown to augment the development of lymphokine-activated killer cells (LAK cells). IL-4 may play an essential role in the pathogenesis of chronic lymphocytic leukemia disease, which is characterized by the accumulation of slow-dividing and long-lived monoclonal B-cells arrested at the intermediate stage of their differentiation by preventing both the death and the proliferation of the malignant B-cells. It protects chronic lymphocytic leukemic B-cells from death by apoptosis and upregulates the expression of a protective gene, BCL-2 (Dancescu et al., Journal of Experimental Medicine 176: 1319-26, 1992).

The determination of IL-6 serum levels may be useful to monitor the activity of myelomas and to calculate tumor cell masses. Low IL-6 serum levels are observed in monoclonal gammopathies and in smoldering myelomas while IL-6 serum levels are markedly increased in patients with progressive disease and also in patients with plasma cell leukemia (Van Oers et al., Ann. Hematology 66: 219-23 (1993).)

The deregulated expression of IL-6 is probably one of the major factor involved in the pathogenesis of a number of diseases (Leger-Ravet et al., Blood, 78: 2923-30, 1991). The excessive overproduction of IL-6 has been observed in various pathological conditions such as rheumatoid arthritis, multiple myeloma, Lennert syndrome (histiocytic lymphoma), Castleman's disease (lymphadenopathy with massive infiltration of plasma cells, hyper gamma-globulinemia, anemia, and enhanced concentrations of acute phase proteins), cardiac myxomas and liver cirrhosis (Hsu et al., Hum. Pathol., 24: 833-9, 1993). The constitutive synthesis of IL-6 by glioblastomas and the secretion of IL-6 into the cerebrospinal fluid may explain the elevated levels of acute phase proteins and immune complexes in the serum (Mule et al. Research Immunology, 143: 777-9, 1991).

IL-6 probably also plays a role in the pathogenesis of chronic polyarthritis because excessive concentrations of IL-6 are found in the synovial fluid. It has been suggested that 16, due to its effects on hematopoietic cells, may be suitable for the treatment of certain types of anemia and thrombocytopenia (Brach et al., International Journal of Clinical and Laboratory Research, 22: 143-51, 1992). Pretreatment with IL-3 and subsequent administration of IL-6 has been shown to increase platelet counts. In combination with other cytokines (for example, IL-2) IL-6 may be useful in the treatment of some tumor types (Dullens et al, In vivo, 5: 567-70,1991).

Very high levels of IL-6 in the cerebrospinal fluid are observed frequently in bacterial and viral meningitis. The detection of elevated concentrations of IL-6 in the urine of transplanted patients may be an early indicator of a graft-versus-host reaction (Kishimoto et al., Blood 74: 1-10, 1990). The detection of IL-6 in the amniotic fluid may be an indication of intra-amniotic infections. In inflammatory intestinal diseases elevated plasma levels of IL-6 may be an indicator of disease status (Wolvekamp et al., Immunology Letters, 24: 1-9, 1990). In patients with mesangioproliferative glomerulonephritis elevated urine levels of IL-6 are also an indicator of disease status (Van Snick et al., Annual Review of Immunology, 8: 253-78, 1990). Monitoring of postoperative serum IL-6 levels may be more helpful than monitoring of C-reactive protein levels for estimation of inflammatory status and early detection of an acute phase reaction (Ohzato et al., Surgery, 111: 201-9, 1992). Serum and urinary IL-6 levels have been shown to be predicting factors of Kawasaki disease activity (Furukawa et al., European Journal of Pediatr,. 151: 44-7, 1992).

It has therefore been suggested that IL-7, alone or in combination with IL-2, may be used as a consolidative immunotherapy for malignancies in patients after autologous bone marrow transplantation.

Participation of IL-7 in the pathogenesis of inflammatory skin diseases and cutaneous T-cell lymphomas is suggested by the growth-promoting effects of IL-7 and its synthesis by keratinocytes.

IL-8 may be of clinical relevance in psoriasis and rheumatoid arthritis. Elevated concentrations are observed in psoriatic scales and this may explain the high proliferation rate observed in these cells (Gillitzer et al., Journal of Investigative Dermatology, 97: 73-9, 1991). IL-8 may be also a marker of different inflammatory processes.

IL-10 has been detected in the sera of a subgroup of patients with active non-Hodgkin's lymphoma. IL-10 levels appear to correlate with a poor survival in patients with intermediate or high-grade non-Hodgkin's lymphoma (Blay et al., Blood, 82: 2169-74, 1993)

The most powerful inducers of IL-12 are bacteria, bacterial products, and parasites. IL-12 has been shown to augment natural killer-cell mediated cytotoxicity in a number of conditions, including patients with hairy cell leukemia (Bigda et al., Leuk. Lymphoma, 10: 121-5, 1993).

IL-13 induces considerable levels of IgM and IgG, but no IgA, in cultures of highly purified surface IgD-positive or total B-cells in the presence of an activated CD4(+) T-cell clone. IL-13 has been shown to inhibit strongly tissue factor expression induced by bacterial lipopolysaccharides and to reduce the pyrogenic effects of IL-1 or TNF, thus protecting endothelial and monocyte surfaces against inflammatory mediator induced procoagulant changes. IL-13 inhibits human immunodeficiency virus type-1 production in primary blood-derived human macrophages in vitro.

IL-13 inhibits human immunodeficiency virus type-1 production in primary blood-derived human macrophages in vitro.

IL-15 stimulates proliferation of T-cells. In addition, IL-15 is also able to induce generation of cytolytic cells and (lymphokine activated killer) LAK cells. IL-15 appears to function as a specific maturation factor for NK-cells. It is also been shown to function as an NK-cell survival factor in vivo. High affinity IL-15 binding has been observed on many lymphoid cell types, including peripheral blood monocytes, and NK-cells.

Interleukin-17 (IL-17) functions as a mediator of angiogenesis that stimulates vascular endothelial cell migration and cord formation and regulates production of a variety of growth factors promoting angiogenesis (Numasaki et al., Blood, 101(7):2620-7, 2002).

Interleukin-18 (IL-18) encodes an inducer of IFN-γ production by T-cells (Okamura et al., Nature, 378: 88-91, 1995; Micallef et al., Cancer Immunology and Immunotherapy, 43: 361-367, 1996) and natural killer cells (Tsutsui et al., J Immunology, 157: 3967-3973, 1996) that is a more potent inducer than IL-12 (Kikkawa et al., Biochemical Biophysical Research Communications, 281(2): 461-7, 2001) have demonstrated that monocytes and macrophages produce large amounts of various IL-18 species.

IL-18 is produced during the acute immune response by macrophages and immature dendritic cells.

IL-18 is considered one of the pro-inflammatory cytokines. An important function of IL-18 is the regulation of functionally distinct subsets of T-helper cells required for cell mediated immune responses (Nakanishi et al., Annual Reviews of Immunology, 19: 423-74, 2001). IL-18 functions as a growth and differentiation factor for Th1 cells.

Granulocyte-Colony Stimulating Factor (G-CSF) is secreted by monocytes, macrophages and neutrophils after cell activation (Demetri et al., Blood, 78: 2791-2808, 1991). It is produced also by stromal cells, fibroblasts, and endothelial cells. Epithelial carcinomas, acute myeloid leukemia cells and various tumor cell lines (bladder carcinomas, medulloblastomas), also express this factor. The synthesis of G-CSF can be induced by bacterial endotoxins, TNF, IL-1, and GM-CSF. Pretreatment with recombinant human G-CSF prior to marrow harvest can improve the graft. One general effect of treatment with G-CSF appears to be a marked reduction of severe infections and episodes of fever, which are normally observed to occur in patients with Kostmann syndrome (Jakubowski et al., New England Journal of Medicine, 320: 38-42, 1989). G-CSF treatment also allows dose intensification with various antitumor drug regimes (Gianni et al., Journal of Clin. Oncol., 10: 1955-62, 1992).

GM-CSF can be employed for the physiological reconstitution of hematopoiesis in all diseases characterized either by an aberrant maturation of blood cells or by a reduced production of leukocytes. GM-CSF can be used also to correct chemotherapy induced cytopenias and to counteract cytopenia-related predisposition to infections and hemorrhages (Fan et al., In vivo, 5: 571-8, 1991). Several studies have demonstrated that the use of GM-CSF enhances tolerance to cytotoxic drug treatment and can be used to prevent dose reductions necessitated by the side effects of cytotoxic drug treatment (Negrin et al., Advances in Pharmacol., 23: 263-296, 1992). At present, GM-CSF represents an important advance in bone marrow transplantation and has become a standard therapy (Armitage et al., Semin. Hematology, 29: s14-8, 1992). GM-CSF enhances the reconstitution of the hematopoietic system in patients undergoing autologous or allogenic bone marrow transplantation and patients with delayed engraftment after bone marrow transplantation (Schuster et al., Infection, 20: S95-9, 1992).

The growth of some tumor cell types in vitro is inhibited by IFN-α which may stimulate also the synthesis of tumor-associated cell surface antigens. In renal carcinomas IFN-α reduces the expression of receptors for EGF. IFN-α also inhibits the growth of fibroblasts and monocytes in vitro. IFN-α also inhibits the proliferation of B-cell in vitro and blocks the synthesis of antibodies. IFN-α also selectively blocks the expression of some mitochondrial genes.

IFN-β enhances the synthesis of the low affinity IgE receptor CD23. In activated monocytes IFN-β induces the synthesis of neopterin. It also enhances serum concentrations of β2-microglobulin. IFN-β selectively inhibits the expression of some mitochondrial genes.

Like the other interferons, IFN-γ can be used as an antiviral and antiparasitic agent (Stuart-Harris et al., Med. Journal of Aust., 156: 869-72, 1992). IFN-γ has been shown to be effective in the treatment of chronic polyarthritis (Machold et al., Ann. Rheum. Dis., 51: 1039-43, 1992). This treatment, which probably involves a modulation of macrophage activities, significantly reduces joint aches and improves various clinical parameters and allows reduction of corticosteroid doses. IFN-γ may be of value in the treatment of opportunistic infections in AIDS patients. It has been shown also to reduce inflammation, clinical symptoms, and eosinophilia in severe atopic dermatitis (Hanifin et al., Am. Acad. Dermatology, 28: 189-97, 1993).

Tumor necrosis factor-α (TNF-α) is secreted by macrophages, monocytes, neutrophils, T-cells, NK-cells following their stimulation by bacterial lipopolysaccharides (Beutler et al., Annual Review of Biochemistry, 57: 505-18, 1988). In contrast to chemotherapeutic drugs TNF-3 specifically attacks malignant cells. Extensive preclinical studies have documented a direct cytostatic and cytotoxic effect of TNF-α against subcutaneous human xenografts and lymph node metastases in nude (immunodeficient) mice, as well as a variety of immunomodulatory effects on various immune effector cells, including neutrophils, macrophages, and T-cells (Gifford et al., Biotherapy, 3: 103-11, 1991).

Macrophage Chemotactic Protein-1 (MCP-1), now called CCL2, activates the tumoricidal activity of monocytes and macrophages in vivo. It regulates the expression of cell surface antigens (CD11c, CD11b) and the expression of cytokines IL-1 and IL-6 (Jiang et al., 1992). CCL2 is a potent activator of human basophils, inducing the degranulation and the release of histamines (Bischoff et al., Journal of Experimental Medicine, 175: 1271-5, 1992). In basophils activated by IL-3, IL-5, or GM-CSF, CCL2 enhances the synthesis of leukotriene C4 (Bischoff et al., European Journal of Immunology, 23: 761-7, 1993). CCL2 has been shown to exhibit biological activities other than chemotaxis. It can induce the proliferation and activation of killer cells known as CHAK (CC-Chemokine activated killer), which are similar to cells activated by IL-2 (LAK cells) (Hora et al., Proc Natl Acad Science USA, 89: 1745-9, 1992). CCL2 is also one of the strongest histamine inducing factors

Vascular endothelial growth factor (VEGF) is important in the pathophysiology of neuronal and other tumors, probably functioning as a potent promoter of angiogenesis for human gliomas. Its synthesis is induced also by hypoxia. The extravasation of cells observed as a response to VEGF may be an important factor determining the colonization of distant sites. Due to its influences on vascular permeability VEGF may be involved also in altering blood-brain-barrier functions under normal and pathological conditions.

Epidermal growth factor (EGF) controls and stimulates the proliferation of epidermal and epithelial cells, including fibroblasts, kidney epithelial cells, human glial cells, ovary granulosa cells, and thyroid cells. EGF alone and also in combination with other cytokines is an important factor mediating wound healing processes. EGF may be a trophic substance for the gastrointestinal mucosa and may play a gastroprotective role due to its ability to stimulate the proliferation of mucosa cells. EGF has been shown to effectively promote healing of ulcers at concentrations that do not inhibit the synthesis of gastric acids.

Exemplary, non-limiting, indications associated with certain cytokines detectable by the methods of the invention are set forth hereinbelow. Each of the exemplary cytokines set forth can be detected on a single molecule basis, or they can be detected by counting event photons or total photons.

In various exemplary embodiments, the disease state is ankylosing spondylitis, and the cytokine detected is selected from the group consisting of CCL4, CCL2, CCL11, EGF, IL-1β, IL-2, IL-5, IL-6, IL-7, CXCL8, IL-10, IL-12, IL-13, IL-15, IL-17, TNF-α, IFNγ, GM-CSF, G-CSF and combinations thereof.

In various exemplary embodiments, the disease state is psoriatic arthritis, and the cytokine detected is selected from the group consisting of GM-CSF, IL-17, IL-2, IL-10, IL-13, IFN-γ, IL-6, CCL4/MIP-1β, CCL11/Eotaxin, EGF, CCL2/MCP-1 and combinations thereof.

In various exemplary embodiments, the disease state is reactive arthritis, and the cytokine detected is selected from the group consisting of IL-12, IFN-γ, IL-1β, IL-13, IL-17, CCL4/MCP-1, TNF-α, IL-4, GM-CSF, CCL11/Eotaxin, EGF, IL-6 and combinations thereof.

In various exemplary embodiments, the disease state is enteropathic arthritis, and the of cytokine detected is selected from the group consisting of CXCL8/IL-8, IL-10, IL-4, G-CSF, CCL2/MCP-1, CCL11/Eotaxin, EGF, IFN-γ, TNF-α and combinations thereof.

In various exemplary embodiments, the disease state is ulcerative colitis (UC), and the cytokine detected is selected from the group consisting of IL-7, CXCL8/IL-8, IFN-γ, TNF-α, EGF, VEGF, IL-1 β and combinations thereof.

In various exemplary embodiments, the disease state is Crohn's Disease (CD), and the cytokine detected is selected from the group consisting of TNF-α, IFN-γ, IL-1β, IL-6, IL-7, IL-13, IL-2, IL-4, GM-CSF, G-CSF, CCL2/MCP-1, EGF, VEGF, CXCL8/IL-8, and combinations thereof.

In various exemplary embodiments, the disease state is rheumatoid arthritis, and the cytokine molecule detected is selected from the group consisting of IFN-γ, IL-1β, TNF-α, G-CSF, GM-CSF, IL-6, IL-4, IL-10, IL-13, IL-5, CCL4/MIP-1β, CCL2/MCP-1, EGF, VEGF, IL-7 and combinations thereof.

In various exemplary embodiments, disease state is systemic lupus erythematosus, the molecule of cytokine detected is selected from the group consisting of IL-10, IL-2, IL-4, IL-6, IFN-γ, CCL2/MCP-1, CCL4/MIP-1β, CXCL8/IL-8, VEGF, EGF, IL-17 and combinations thereof.

In various exemplary embodiments, disease state is Familial Mediterranean Fever (FMF), and the molecule of cytokine detected is selected from the group consisting of G-CSF, IL-2, IFN-γ, TNF-α, IL-1β, CXCL8/IL-8 and combinations thereof.

In various exemplary embodiments, disease state is amyotrophic lateral sclerosis (ALS), and the molecule of cytokine detected is selected from the group consisting of CCL2/MIP-1β, CXCL8/IL-8, IL-12, IL-1β, VEGF, IL-13 and combinations thereof.

In various exemplary embodiments, disease state is Irritable Bowel Syndrome (IBS), and the molecule of cytokine detected is selected from the group consisting of TNF-α, IFN-γ, IL-1β, IL-6, IL-7, GM-CSF, G-CSF, CCL2/MCP-1, CXCL8/IL-8 and combinations thereof.

In various exemplary embodiments, disease state is Juvenile Rheumatoid Arthritis (JRA), and the molecule of cytokine detected is selected from the group consisting of IFN-γ, IL-1β, TNF-α, G-CSF, GM-CSF, IL-6, IL-4, IL-10, IL-13, IL-5, IL-7 and combinations thereof.

In various exemplary embodiments, disease state is Sjogren's Syndrome, and the molecule of cytokine detected is selected from the group consisting of CCL2/MCP-1, IL-12, CXCL8/IL-8, CCL11/Eotaxin, TNFα, IL-2, IFNα, IL-15, IL-17, IL-1α, IL-1β, IL-6, GM-CSF and combinations thereof.

In various exemplary embodiments, disease state is early arthritis, and the molecule of cytokine detected is selected from the group consisting of CCL4/MIP i 3, CXCL8/IL-8, IL-2, IL-12, IL-17, IL-13, TNFα, IL-4, IL-5, IL-10 and combinations thereof.

In various exemplary embodiments, disease state is neuroinflammation, and the molecule of cytokine detected is selected from the group consisting of CCL2/MCP-1, IL-12, GM-CSF, G-CSF, M-CSF, IL-6, IL-17 and combinations thereof.

In various embodiments, specific cytokines measured in the assays of the invention include, but are not limited to, IL-1α, IL-1β, IL-2, IL-4, IL-8, IL-12, IL-21, G-CSF, GM-CSF, hTNFα, mTNFα, IFNγ, and hVEGF. According to one aspect of the invention, analytes are advantageously measured in a sample obtained via a non-surgically invasive collection technique, such as in a blood, serum, plasma, fecal, or urine sample from a patient having, or suspected to have a disease in which a cytokine is either implicated or its detection and/or quantification produces diagnostic or prognostic yield.

In some embodiments, the method includes determining the level of more than one, e.g., 2, 3, 4, 5, 6, 7, 8, 9, 10, 11, 12, 13, 14, 15, 16, 17, 18, 19 or 20 or more cytokines using the detection methods of the present invention.

In an exemplary embodiment, the invention provides a method as set forth above in which the single molecule of a detectable species correlates to a single molecule of an analyte which is a member selected from a cytokine and a growth factor, to which said detectable species specifically binds. In various embodiments, the assay is capable of detecting said detectable species in an amount of less than or equal to about 5 pg/mL. In certain embodiments, the assay detects a single molecule of the detectable species in an amount of less than or equal to about 2 pg/mL.

The present invention also provides assays of robust dynamic range. In exemplary embodiments, the dynamic detection range is at least about 2 log, preferably at least about 3 log and more preferably at least about 4 log.

The assays of the invention are highly sensitive for cytokines and other disease markers. For example, in one embodiment, the method provides an assay capable of detecting at least one cytokine in an amount less than or equal to about 5 pg/mL, 4 pg/mL, 3 pg/mL, 2 pg/mL, 1 pg/mL, 0.5 pg/mL, and even less than or equal to about 0.1 pg/mL.

In various embodiments, the invention provides a method of detecting IL-2 at a concentration of less than about 2 pg/mL, less than about 1 pg/mL, less than about 0.8 pg/mL, less than about 0.6 pg/mL, less than about 0.4 pg/mL or less than about 0.3 pg/mL. In various embodiments, the amount detected corresponds to an amount of IL-2 found in a healthy subject.

In an exemplary embodiment, the invention provides an assay for IL-2 with sensitivity for IL-2 (limit of detection (LOD)) of less than or equal to about 0.1 pg/mL, e.g., about 0.07 pg/mL. In various embodiments, the assay of the invention quantifies IL-2 in plasma in a range of from about 0.1 pg/mL to about 2 pg/mL, preferably from about 0.16 pg/mL to about 1.5 pg/mL. An exemplary assay has a LOD for IL-2 of less than or equal to about 0.1 pg/mL (e.g., about 0.07 pg/mL) and quantifies IL-2 in plasma within a range of from about 0.1 pg/mL to about 2 pg/mL, preferably from about 0.16 pg/mL to about 1.5 pg/mL. In one embodiment, the plasma is from a healthy subject.

In various embodiments, the invention provides a method of detecting IL-5 at a concentration of less than about 20 pg/mL, less than about 10 pg/mL, less than about 8 pg/mL, less than about 6 pg/mL or less than about 4 pg/mL. In various embodiments, the amount detected corresponds to an amount of IL-2 found in a healthy subject.

In an exemplary embodiment, the invention provides an assay for IL-5 with sensitivity for IL-5 (limit of detection (LOD)) of less than or equal to about 1.2 pg/mL, e.g., about 0.9 pg/mL. In various embodiments, the assay of the invention quantifies IL-5 in plasma in a range of from about 1.5 pg/mL to about 19.5 pg/mL, preferably from about 2.0 pg/mL to about 19 pg/mL. An exemplary assay has a LOD for IL-5 of less than or equal to about 1.2 pg/mL (e.g., about 0.9 pg/mL) and quantifies IL-5 in plasma within a range of from about 1.5 pg/mL to about 19.5 pg/mL, preferably from about 2.0 pg/mL to about 19 pg/mL. In one embodiment, the plasma is from a healthy subject.

In various embodiments, the invention provides a method of detecting IL-7 at a concentration of less than about 3 pg/mL, less than about 2 pg/mL, less than about 1 pg/mL, less than about 0.8 pg/mL or less than about 0.6 pg/mL. In various embodiments, the amount detected corresponds to an amount of IL-2 found in a healthy subject.

In an exemplary embodiment, the invention provides an assay for IL-7 with sensitivity for IL-7 (limit of detection (LOD)) of less than or equal to about 0.02 pg/mL, e.g., about 0.012 pg/mL. In various embodiments, the assay of the invention quantifies IL-7 in plasma in a range of from about 0.5 pg/mL to about 3 pg/mL, preferably from about 0.4 pg/mL to about 2.7 pg/mL. An exemplary assay has a LOD for IL-7 of less than or equal to about 0.02 pg/mL (e.g., about 0.012 pg/mL) and quantifies IL-7 in plasma within a range of from about 0.5 pg/mL to about 3 pg/mL, preferably from about 0.4 pg/mL to about 2.7 pg/mL. In one embodiment, the plasma is from a healthy subject.

In various embodiments, the invention provides a method of detecting IL-21 at a concentration of less than about 12 pg/mL, less than about 10 pg/mL, less than about 8 pg/mL, less than about 6 pg/mL or less than about 4 pg/mL. In various embodiments, the amount detected corresponds to an amount of IL-2 found in a healthy subject.

In an exemplary embodiment, the invention provides an assay for IL-21 with sensitivity for IL-21 (limit of detection (LOD)) of less than or equal to about 0.1 pg/mL, e.g., about 0.06 pg/mL. In various embodiments, the assay of the invention quantifies IL-21 in plasma in a range of from about 2 pg/mL to about 10 pg/mL, preferably from about 1.8 pg/mL to about 9.3 pg/mL. An exemplary assay has a LOD for IL-21 of less than or equal to about 0.1 pg/mL (e.g., about 0.06 pg/mL) and quantifies IL-21 in plasma within a range of from about 2 pg/mL to about 10 pg/mL, preferably from about 1.8 pg/mL to about 9.3 pg/mL. In one embodiment, the plasma is from a healthy subject.

In various embodiments, the invention provides a method of detecting IL-1b at a concentration of less than about 12 pg/mL, less than about 0.4 pg/mL, less than about 0.3 pg/mL, less than about 0.2 pg/mL or less than about 0.1 pg/mL. In various embodiments, the amount detected corresponds to an amount of IL-1b found in a healthy subject.

In an exemplary embodiment, the invention provides an assay for IL-21 with sensitivity for IL-1b (limit of detection (LOD)) of less than or equal to about 0.1 pg/mL, e.g., about 0.06 pg/mL. In various embodiments, the assay of the invention quantifies IL-1b in plasma within a range of from about 0.03 pg/mL to about 0.3 pg/mL, preferably from about 0.05 pg/mL to about 0.2 pg/mL. An exemplary assay has a LOD for IL-1b of less than or equal to about 0.1 pg/mL (e.g., about 0.06 pg/mL) and quantifies IL-1b in plasma within a range of from about 0.03 pg/mL to about 0.3 pg/mL, preferably from about 0.05 pg/mL to about 0.2 pg/mL. In one embodiment, the plasma is from a healthy subject.

In various embodiments, the invention provides a method of detecting IFNg at a concentration of less than about 7 pg/mL, less than about 6 pg/mL, less than about 5 pg/mL, less than about 4 pg/mL or less than about 3 pg/mL. In various embodiments, the amount detected corresponds to an amount of IL-1b found in a healthy subject.

In an exemplary embodiment, the invention provides an assay for IFNg with sensitivity for IFNg (limit of detection (LOD)) of less than or equal to about 0.1 pg/mL, e.g., about 0.09 pg/mL. In various embodiments, the assay of the invention quantifies IFNg in plasma within a range of from about 7 pg/mL to about 2 pg/mL, preferably from about 6.7 pg/mL to about 2.6 pg/mL. An exemplary assay has a LOD for IFNg of less than or equal to about 0.1 pg/mL (e.g., about 0.06 pg/mL) and quantifies IFNg in plasma within a range of from about 7 pg/mL to about 2 pg/mL, preferably from about 6.7 pg/mL to about 2.6 pg/mL. In one embodiment, the plasma is from a healthy subject.

In various embodiments, the invention provides a method of detecting IL-6 at a concentration of less than about 9 pg/mL, less than about 6 pg/mL, less than about 3 pg/mL, less than about 2 pg/mL or less than about 1 pg/mL. In various embodiments, the amount detected corresponds to an amount of IL-1b found in a healthy subject.

In an exemplary embodiment, the invention provides an assay for IL-6 with sensitivity for IL-6 (limit of detection (LOD)) of less than or equal to about 0.01 pg/mL, e.g., about 0.003 pg/mL. In various embodiments, the assay of the invention quantifies IL-6 in plasma within a range of from about 9 pg/mL to about 0.5 pg/mL, preferably from about 8.9 pg/mL to about 0.48 pg/mL. An exemplary assay has a LOD for IL-6 of less than or equal to about 0.01 pg/mL (e.g., about 0.06 pg/mL) and quantifies IL-6 in plasma within a range of from about 9 pg/mL to about 0.5 pg/mL, preferably from about 8.9 pg/mL to about 0.48 pg/mL. In one embodiment, the plasma is from a healthy subject.

In various embodiments, the invention provides a method of detecting IL-17A at a concentration of less than about 1 pg/mL, less than about 0.8 pg/mL, less than about 0.6 pg/mL, less than about 0.4 pg/mL or less than about 0.1 pg/mL. In various embodiments, the amount detected corresponds to an amount of IL-17A found in a healthy subject.

In an exemplary embodiment, the invention provides an assay for IL-17A with sensitivity for IL-17A (limit of detection (LOD)) of less than or equal to about 0.1 pg/mL, e.g., about 0.08 pg/mL. In various embodiments, the assay of the invention quantifies IL-17A in plasma within a range of from about 0.7 pg/mL to about 0.05 pg/mL, preferably from about 0.6 pg/mL to about 0.06 pg/mL. An exemplary assay has a LOD for IL-17A of less than or equal to about 0.1 pg/mL (e.g., about 0.08 pg/mL) and quantifies IL-17A in plasma within a range of from about 0.7 pg/mL to about 0.05 pg/mL, preferably from about 0.6 pg/mL to about 0.06 pg/mL. In one embodiment, the plasma is from a healthy subject.

In various embodiments, the invention provides a method of detecting TNFa at a concentration of less than about 3 pg/mL, less than about 2 pg/mL, less than about 1 pg/mL, less than about 0.8 pg/mL or less than about 0.6 pg/mL. In various embodiments, the amount detected corresponds to an amount of TNFa found in a healthy subject.

In an exemplary embodiment, the invention provides an assay for TNFa with sensitivity for TNFa (limit of detection (LOD)) of less than or equal to about 0.01 pg/mL, e.g., about 0.006 pg/mL. In various embodiments, the assay of the invention quantifies TNFa in plasma within a range of from about 0.3 pg/mL to about 0.5 pg/mL, preferably from about 2.7 pg/mL to about 0.6 pg/mL. An exemplary assay has a LOD for TNFa of less than or equal to about 0.01 pg/mL (e.g., about 0.08 pg/mL) and quantifies TNFa in plasma within a range of from about 0.3 pg/mL to about 0.5 pg/mL, preferably from about 2.7 pg/mL to about 0.6 pg/mL. In one embodiment, the plasma is from a healthy subject.

In various embodiments, the invention provides a method of detecting IL-4 at a concentration of less than about 1.5 pg/mL, less than about 1 pg/mL, less than about 0.75 pg/mL, less than about 0.5 pg/mL or less than about 0.4 pg/mL. In various embodiments, the amount detected corresponds to an amount of IL-4 found in a healthy subject.

In an exemplary embodiment, the invention provides an assay for IL-4 with sensitivity for IL-4 (limit of detection (LOD)) of less than or equal to about 0.15 pg/mL, e.g., about 0.11 pg/mL. In various embodiments, the assay of the invention quantifies IL-4 in plasma within a range of from about 1 pg/mL to about 0.1 pg/mL, preferably from about 0.8 pg/mL to about 0.2 pg/mL. An exemplary assay has a LOD for IL-4 of less than or equal to about 0.15 pg/mL (e.g., about 0.11 pg/mL) and quantifies IL-4 in plasma within a range of from about 1 pg/mL to about 0.1 pg/mL, preferably from about 0.8 pg/mL to about 0.2 pg/mL. In one embodiment, the plasma is from a healthy subject.

In various embodiments, the invention provides a method of detecting IL-1a at a concentration of less than about 1 pg/mL, less than about 5 pg/mL, less than about 0.3 pg/mL, less than about 0.2 pg/mL or less than about 0.1 pg/mL. In various embodiments, the amount detected corresponds to an amount of IL-1a found in a healthy subject.

In an exemplary embodiment, the invention provides an assay for IL-1a with sensitivity for IL-1a (limit of detection (LOD)) of less than or equal to about 0.1 pg/mL, e.g., about 0.06 pg/mL. In various embodiments, the assay of the invention quantifies IL-1a in plasma within a range of from about 0.6 pg/mL to about 0.05 pg/mL, preferably from about 0.5 pg/mL to about 0.07 pg/mL. An exemplary assay has a LOD for IL-1a of less than or equal to about 0.1 pg/mL (e.g., about 0.06 pg/mL) and quantifies IL-1a in plasma within a range of from about 0.6 pg/mL to about 0.05 pg/mL, preferably from about 0.5 pg/mL to about 0.07 pg/mL. In one embodiment, the plasma is from a healthy subject.

In various embodiments, the invention provides a method of detecting GM-CSF at a concentration of less than about 2 pg/mL, less than about 1.5 pg/mL, less than about 1 pg/mL, less than about 0.8 pg/mL or less than about 0.5 pg/mL. In various embodiments, the amount detected corresponds to an amount of GM-CSF found in a healthy subject.

In an exemplary embodiment, the invention provides an assay for GM-CSF with sensitivity for GM-CSF (limit of detection (LOD)) of less than or equal to about 0.05 pg/mL, e.g., about 0.03 pg/mL. In various embodiments, the assay of the invention quantifies GM-CSF in plasma within a range of from about 1.3 pg/mL to about 0.2 pg/mL, preferably from about 1.1 pg/mL to about 0.3 pg/mL. An exemplary assay has a LOD for GM-CSF of less than or equal to about 0.05 pg/mL (e.g., about 0.06 pg/mL) and quantifies GM-CSF in plasma within a range of from about 1.3 pg/mL to about 0.2 pg/mL, preferably from about 1.1 pg/mL to about 0.3 pg/mL. In one embodiment, the plasma is from a healthy subject.

In various embodiments, the invention provides a method of detecting IL-12 at a concentration of less than about 2 pg/mL, less than about 1 pg/mL, less than about 0.5 pg/mL, less than about 0.2 pg/mL or less than about 0.1 pg/mL. In various embodiments, the amount detected corresponds to an amount of IL-12 found in a healthy subject.

In an exemplary embodiment, the invention provides an assay for IL-12 with sensitivity for IL-12 (limit of detection (LOD)) of less than or equal to about 0.008 pg/mL, e.g., about 0.01 pg/mL. In various embodiments, the assay of the invention quantifies IL-12 in plasma within a range of from about 1.2 pg/mL to about 0.05 pg/mL, preferably from about 1 pg/mL to about 0.08 pg/mL. An exemplary assay has a LOD for IL-12 of less than or equal to about 0.008 pg/mL (e.g., about 0.01 pg/mL) and quantifies IL-12 in plasma within a range of from about 1.2 pg/mL to about 0.05 pg/mL, preferably from about 1 pg/mL to about 0.08 pg/mL. In one embodiment, the plasma is from a healthy subject.

In various embodiments, the invention provides a method of detecting G-CSF at a concentration of less than about 150 pg/mL, less than about 100 pg/mL, less than about 80 pg/mL, less than about 60 pg/mL or less than about 40 pg/mL. In various embodiments, the amount detected corresponds to an amount of G-CSF found in a healthy subject.

In an exemplary embodiment, the invention provides an assay for G-CSF with sensitivity for G-CSF (limit of detection (LOD)) of less than or equal to about 0.2 pg/mL, e.g., about 0.12 pg/mL. In various embodiments, the assay of the invention quantifies G-CSF in plasma within a range of from about 110 pg/mL to about 30 pg/mL, preferably from about 97 pg/mL to about 25 pg/mL. An exemplary assay has a LOD for G-CSF of less than or equal to about 0.2 pg/mL (e.g., about 0.01 pg/mL) and quantifies G-CSF in plasma within a range of from about 110 pg/mL to about 30 pg/mL, preferably from about 97 pg/mL to about 25 pg/mL. In one embodiment, the plasma is from a healthy subject.

In various embodiments, the invention provides a method of detecting IL-22 at a concentration of less than about 50 pg/mL, less than about 40 pg/mL, less than about 30 pg/mL, less than about 20 pg/mL or less than about 10 pg/mL. In various embodiments, the amount detected corresponds to an amount of IL-22 found in a healthy subject.

In an exemplary embodiment, the invention provides an assay for IL-22 with sensitivity for IL-22 (limit of detection (LOD)) of less than or equal to about 0.03 pg/mL, e.g., about 0.02 pg/mL. In various embodiments, the assay of the invention quantifies IL-22 in plasma within a range of from about 40 pg/mL to about 3 pg/mL, preferably from about 39 pg/mL to about 4 pg/mL. An exemplary assay has a LOD for IL-22 of less than or equal to about 0.03 pg/mL (e.g., about 0.01 pg/mL) and quantifies IL-22 in plasma within a range of from about 40 pg/mL to about 3 pg/mL, preferably from about 39 pg/mL to about 4 pg/mL. In one embodiment, the plasma is from a healthy subject.

In various embodiments, the invention provides a method of detecting IL-10 at a concentration of less than about 40 pg/mL, less than about 30 pg/mL, less than about 20 pg/mL, or less than about 10 pg/mL. In various embodiments, the amount detected corresponds to an amount of IL-10 found in a healthy subject.

In an exemplary embodiment, the invention provides an assay for IL-10 with sensitivity for IL-10 (limit of detection (LOD)) of less than or equal to about 0.5 pg/mL, e.g., about 0.4 pg/mL. In various embodiments, the assay of the invention quantifies IL-10 in plasma within a range of from about 40 pg/mL to about 8 pg/mL, preferably from about 35 pg/mL to about 10 pg/mL. An exemplary assay has a LOD for IL-10 of less than or equal to about 0.5 pg/mL (e.g., about 0.4 pg/mL) and quantifies IL-10 in plasma within a range of from about 40 pg/mL to about 8 pg/mL, preferably from about 35 pg/mL to about 10 pg/mL. In one embodiment, the plasma is from a healthy subject.

In various embodiments, the invention provides a method of detecting MIP-1a at a concentration of less than about 70 pg/mL, less than about 60 pg/mL, less than about 50 pg/mL, less than about 40 pg/mL, or less than about 30 pg/mL. In various embodiments, the amount detected corresponds to an amount of MIP-1a found in a healthy subject.

In an exemplary embodiment, the invention provides an assay for MIP-1a with sensitivity for MIP-1a (limit of detection (LOD)) of less than or equal to about 0.3 pg/mL, e.g., about 0.2 pg/mL. In various embodiments, the assay of the invention quantifies MIP-1a in plasma within a range of from about 65 pg/mL to about 25 pg/mL, preferably from about 63 pg/mL to about 22 pg/mL. An exemplary assay has a LOD for MIP-1a of less than or equal to about 0.3 pg/mL (e.g., about 0.2 pg/mL) and quantifies MIP-1a in plasma within a range of from about 65 pg/mL to about 25 pg/mL, preferably from about 63 pg/mL to about 22 pg/mL. In one embodiment, the plasma is from a healthy subject.

Moreover, the detection methods of the invention provides robust assays with a broad dynamic range. For example, the method of the invention provide assays with dynamic ranges of at least 1 log, 1.5 log, 2 log, 2.5 log, 3 log, 3.5 log and up to 4 log and greater than at least 4 log.

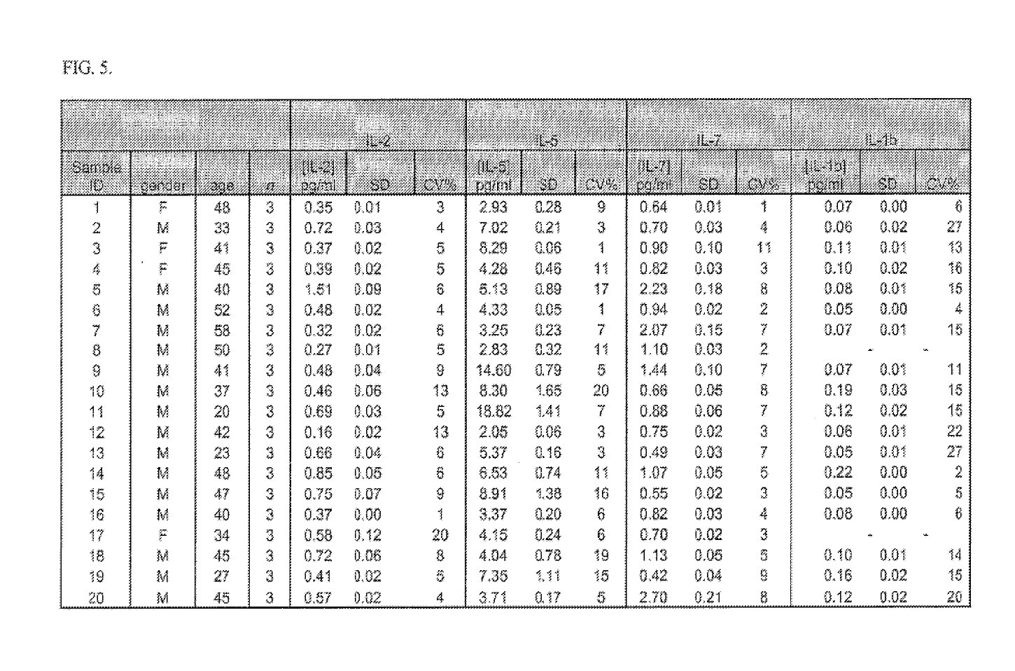

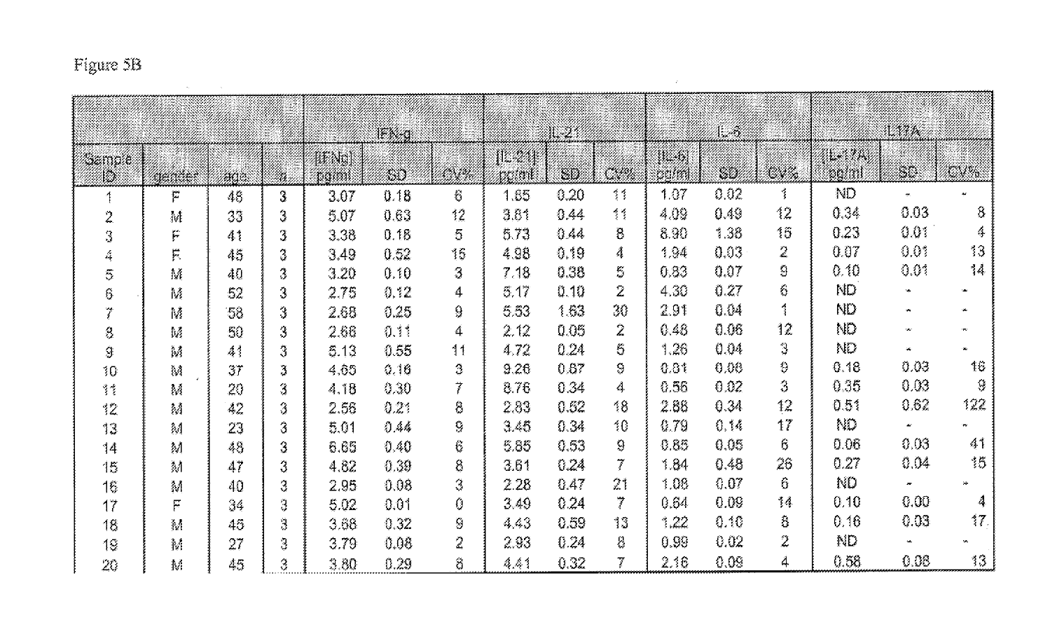

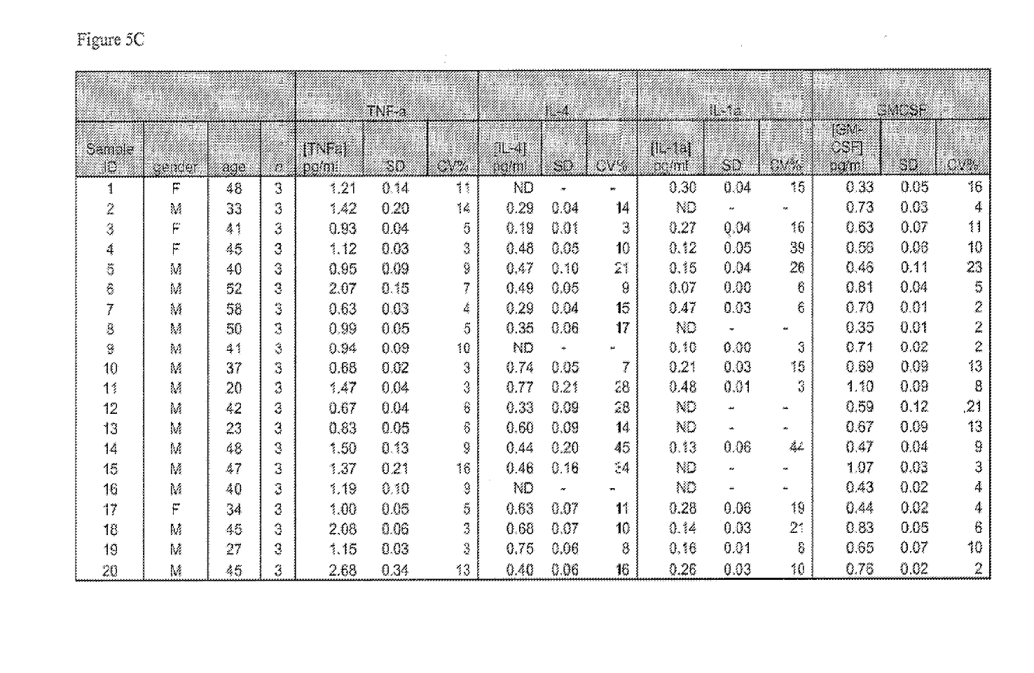

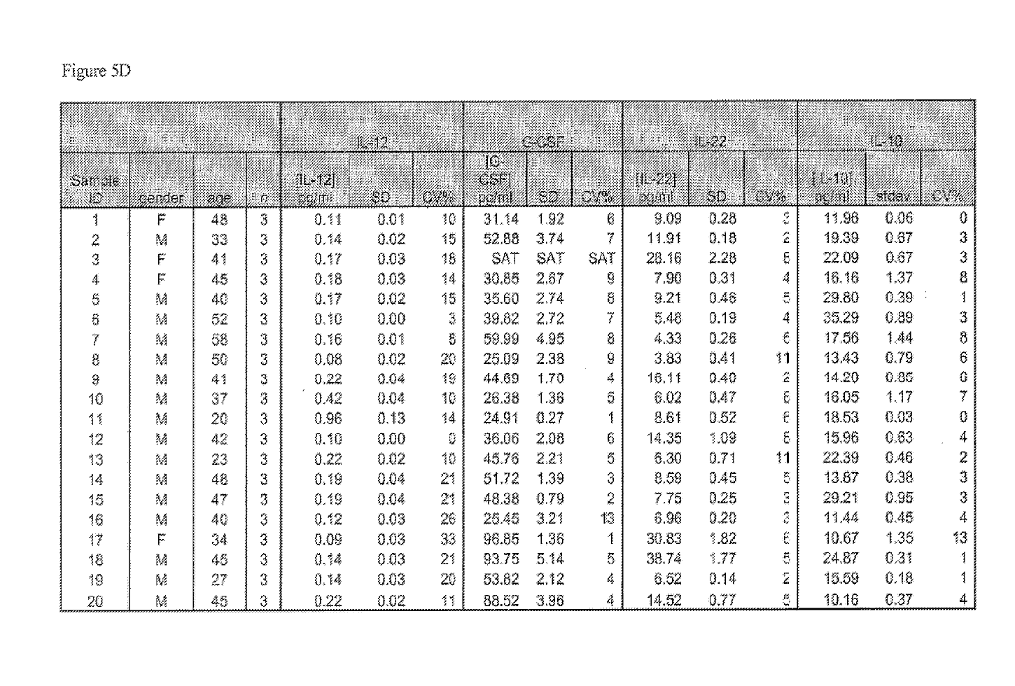

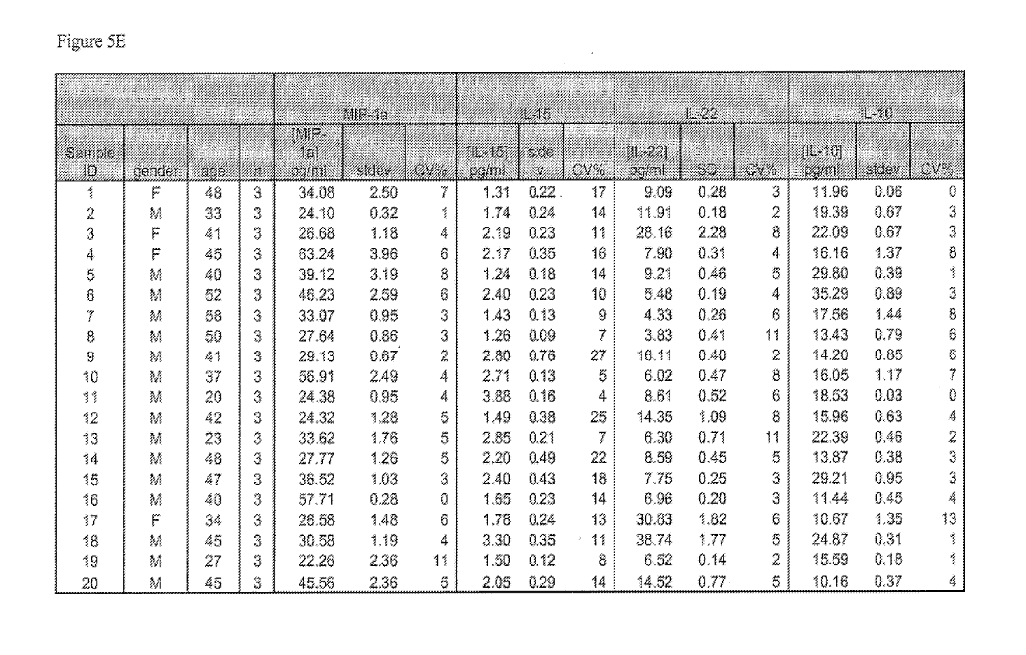

In an exemplary embodiment, the invention provides a method of determining whether a patient is healthy or is affected by a disease, syndrome or other malady. The method includes preparing a sample of an analyte and detecting a detectable species corresponding to a single molecule of the analyte as set forth herein. In various embodiments, the cytokine detected is a member selected from IL-2, IL-5, IL-7, IL-21 and a combination thereof (FIG. 5). The measured concentration of cytokine is compared to a concentration reference standard of cytokine, or threshold cytokine concentration corresponding to a healthy individual. If the measured concentration of cytokine in the sample diverges by more than a pre-determined amount from that in the reference standard or from the threshold healthy cytokine value, this is indicative of a subject affected by a disease, syndrome or other malady.

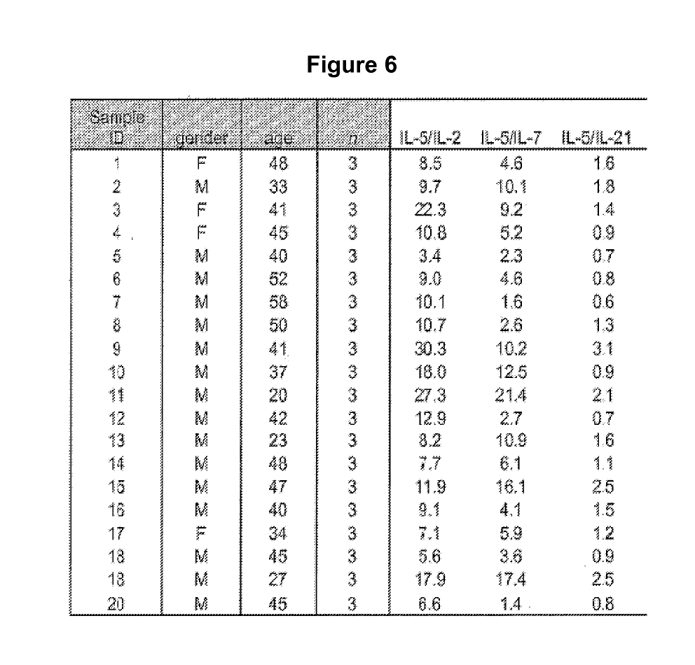

In a variation on these embodiments, a ratio of a first cytokine concentration and a second cytokine concentration is taken. The ratio is then compared to a reference ratio (e.g., a ratio corresponding to cytokine concentrations in a healthy subject) and if the value of the ratio of cytokine concentration measured in the sample diverges by more than a predetermined amount from that of the reference ratio, this is indicative of a subject affected by a disease, syndrome or other malady. In exemplary embodiments, the ratio is selected from [IL-5]/[IL-2], [IL-5]/[IL-7], [IL-5]/[IL-21] and combinations thereof (FIG. 6). In exemplary embodiments, the ratio of [IL-5]/[IL-2] indicative of a healthy subject is about 30 or less, about 20 or less, about 15 or less, about 10 or less or about 8 or less. In exemplary embodiments, the ratio of [IL-5]/[IL-7] indicative of a healthy subject is about 25 or less, about 15 or less, about 10 or less, about 5 or less or about 3 or less. In various embodiments, the ratio of [IL-5]/[IL-21] indicative of a healthy subject is about 5 or less, about 4 or less, about 3 or less, about 2 or less or about 1 or less.

### Sample Preparation

Various embodiments of the invention are based on the recognition that certain sample parameters produce assays with enhanced sensitivity and dynamic range. In general, the prior art recognizes advantages from preparing assay samples in small volumes, thereby minimizing transfer losses contributing to a loss in assay sensitivity. The present invention provides methods in which the sensitivity of assays is increased through use of volumes larger than those generally recognized in the art as useful. Among the assay characteristics altered by the use of higher liquid volumes is the deleterious effect on sensitivity due to non-specific matrix effect binding of non-analyte species within an assay mixture. In an exemplary embodiment, the present invention provides an assay mixture of a volume sufficient to provide matrix binding effects of a lower magnitude than an identical assay mixture at a lower volume.

For example, in certain embodiments, forming the complex between the analyte and the capture species is performed in an amount of buffer solution of at least about 25mL, e.g., from about 25 μL to about 1000 μL, from about 40 μL to about 800 mL, from about 60 μL to about 500 μL and from about 75 μL to about 300 μL.

Similarly, the inventors have recognized that forming the complex between the magnetic particle immobilized capture species and the analyte using a particular weight range of magnetic particle immobilized capture species enhances the sensitivity and dynamic range of the assays. In an exemplary embodiment, at least about 0.1 μg, e.g., from about 0.1 μg to about 40 μg, from about 0.5 μg to about 20 μg, and from about 1.0 to about 10 μg of the magnetic particles conjugated to the capture species are used.

In various embodiments, the sensitivity of an assay of the invention is enhanced by controlling the magnitude of the volume utilized to elute the detectable species from the complex comprising the analyte, the capture species and the detectable species. Thus, in one embodiment, the elution volume of the detectable species is smaller than the original sample size. In various embodiments, the elution volume is from about 2-times to about 100-times smaller than the original sample volume, for example from about 2-times to about 20-times smaller than the original sample volume. In various embodiments, the elution volume is about 5-times to about 15-times smaller than the original sample volume.

In an exemplary embodiment, a sample size of about 200 μL is reduced during a method of the invention to an elution volume from about 2-times to about 20-times smaller than the original sample volume. In another exemplary embodiment, a sample size of about 100 μL is reduced during a method of the invention to an elution volume from about 2-times to about 20-times smaller than the original sample volume. In other exemplary embodiments, an original sample volume of from about 100 μL to about 200 μL is reduced to a volume of from about 1 μL to about 40 μL, for example, from about 5 μL to about 30 μL, or from about 10 μL to about 20 μL.

In an exemplary embodiment, the complex between the capture species and the analyte is formed in a mixture comprising from about 0.1 g to about 40 μg of a plurality of the magnetic particles to about 25 μL to about 1000 μL of a buffer solution. In certain embodiments, the complex is formed by contacting from about 0.5 μg to about 25 μg of the plurality of magnetic particles with from about 100 μL to about 1000 μL of a plasma sample.

In one embodiment, the method provided is analogous to a sandwich assay. Thus, monoclonal antibodies are used as binding partners. The primary antibody is linked to a magnetic particle to serve as capture antibody (“capture species”). The sample is then added and single molecules having the epitope recognized by the antibody bind to the antibody on the surface. Unbound analyte molecules are washed away leaving essentially only specifically bound analyte molecules. The bound analyte molecule/antibody are reacted with a detection antibody (“detectable species”) containing a detectable label. After incubating to allow reaction between the detection antibodies and analyte molecules, non-specifically bound detection antibodies are washed away. In an exemplary embodiment, the analyte-antibody-detectable species complex is rendered essentially free from contaminating unbound detectable species by transferring the magnetic particles to a vessel in which other components of the assay, particularly the detectable species, are absent. The single molecule and detection antibody are separated using an elution buffer and the detectable species is in the single molecule analyzer. Alternatively, only the label bound to the detectable species can be released and detected, thereby indirectly detecting the single molecule. As will be appreciated by those of skill in the art, the use of antibodies as capture and/or detectable species is for clarity of illustration and essentially any other capture or detectable species can be substituted for the antibodies incorporated in this example without departing from the spirit of the instant invention. Moreover, those of skill will understand that other methods for separating unbound detectable species from the analyte-antibody-detectable species complex are within the scope of the instant invention.

In various embodiments, the precision of the instant method is enhanced by use of samples of repeatable composition. Accordingly, in an exemplary embodiment, the samples are either separated into regions of higher and lower concentrations of sample components, or are homogenized to produce a uniform sample. In one embodiment, the sample is a serum sample separated into concentration gradients of its various components. For example, one sample is centrifuged to produce a sample detectably separated into three or more layers. In one example, the sample is separated into three detectable layers, including a lipid layer (e.g., an upper lipid layer), a middle layer containing the analyte of interest, and a layer containing fibrin clot (e.g., a lower fibrin layer). In various exemplary embodiments, the analyte of interest resides in the middle layer. The invention provides an assay in which the sample is withdrawn from the middle layer

### Detection

The methods described herein allow single molecules to be enumerated as they pass through the interrogation spaces one at a time. The concentration of the sample can be determined from the number of single molecules counted and the volume of sample passing though the interrogation space in a set length of time. In the case where an interrogation space encompasses the entire cross-section of the sample stream, only the number of single molecules counted and the volume passing through a cross-section of the sample stream in a set length of time are needed to calculate the concentration the sample. When an interrogation space is smaller than the sample stream, the concentration of the single molecule can be determined by interpolating from a standard curve generated with a control sample of standard concentration. In various embodiments, the concentration of the single molecule can be determined by comparing the measured single molecules to an internal single molecule standard. Concentration can be deduced from the number of single molecules counted. For example, knowing the sample dilution, one can calculate the concentration of single molecules in the starting sample.

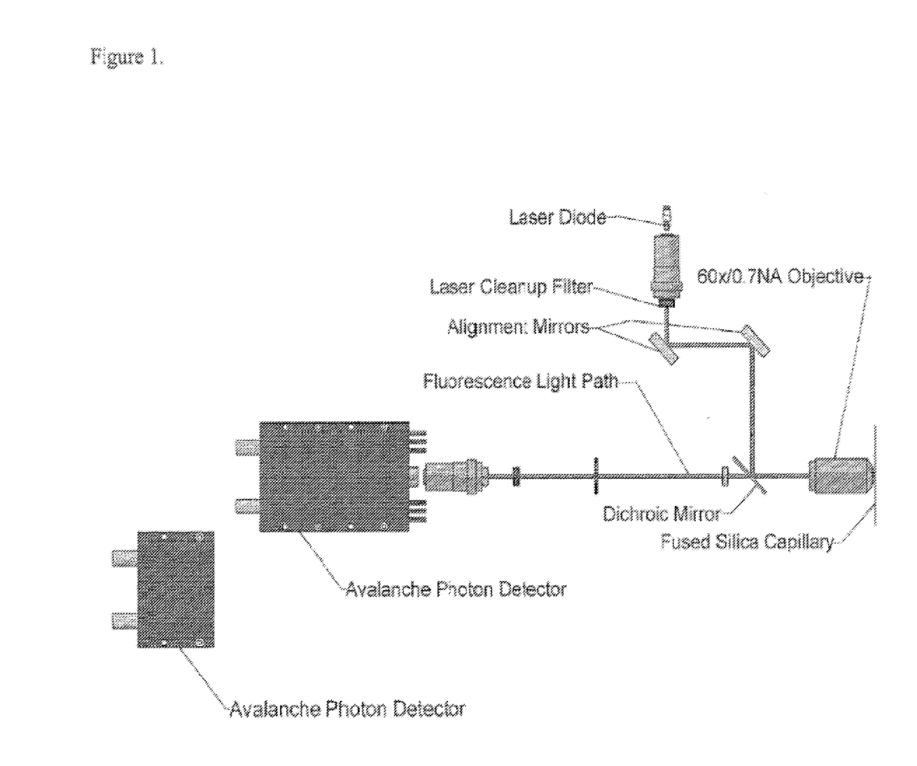

An exemplary device for detecting single molecules according to the methods of the invention is disclosed in commonly owned, published U.S. Patent Application No.s 20060078998, and 20080171352. A further example of an appropriate system is the optical system made by Singulex, Inc., FIG. 1.

In an exemplary embodiment, detection of a single molecule utilizes a single molecule analyzer. An exemplary single molecule analyzer includes an electromagnetic radiation source for emitting electromagnetic radiation. The source is optionally a continuous wave radiation source. The analyzer also includes a first interrogation space positioned to receive electromagnetic radiation emitted from the electromagnetic radiation source. The interrogation space is optionally adjustable and in some embodiments has as volume of from about 0.02 pL to about 300 pL, from about 0.05 pL to about 50 pL, or from about 0.1 pL to about 25 pL.

The analyzer also includes a first electromagnetic radiation detector operably connected to the first interrogation space to measure a first electromagnetic characteristic of the single molecule of said detectable species. In various embodiments, the analyzer also includes a sampling system capable of automatically sampling at least one sample and providing a fluid communication between a sample containers and said first interrogation space. In certain embodiments, the analyzer system further includes a sample recovery system in fluid communication with the first interrogation space. Ideally, the recovery system is capable of recovering substantially all the sample. The analyzer can optionally include a sample preparation system.

A feature that contributes to the extremely high sensitivity of the methods of the invention is the method of detecting and counting detectable species, which, in some embodiments, are attached to single molecules to be detected or correspond to a single molecule to be detected. Briefly, in an exemplary embodiment, the processing sample flowing through the detection apparatus is effectively divided into a series of detection events, by subjecting a given interrogation space to EM radiation from a laser that emits light at an appropriate excitation wavelength for the fluorescent moiety used in the label for a predetermined period of time, and detecting photons emitted during that time. The signals detected by the detector are divided into arbitrary time segments (bins) each having a pre-selected length of time (bin width). Although other bin widths may be used without departing from the scope of the present invention, in one embodiment the bin widths are selected in the range of about 10 μs to about 5 ms. An exemplary bin width is 1 ms. The number of signals contained in each segment is established.

Each predetermined period of time is a “bin.” If the total number of photons detected in a given bin exceeds a predetermined threshold level, a detection event is registered for that bin, i.e., a label has been detected. If the total number of photons is not at the predetermined threshold level, no detection event is registered. In some embodiments, processing sample concentration is dilute enough that, for a large percentage of detection events, the detection event represents only one detectable species passing through the window, which corresponds to a single molecule of interest in the original sample, that is, few detection events represent more than one label in a single bin. In some embodiments, further refinements are applied to allow greater concentrations of label in the processing sample to be detected accurately, i.e., concentrations at which the probability of two or more labels being detected as a single detection event is no longer insignificant.

In an exemplary embodiment, the method provides detection by counting single molecules only. The method of the invention can also include counting multiple copies of a detectable species in a single bin. Thus, in another embodiment, the invention provides for detection by counting a member selected from photons from single molecules, event photons, total photons and a combination thereof.

In one embodiment, the detected signal is first analyzed to determine the noise level and signals are selected above a threshold prior to utilizing the data. In one embodiment, the noise level is determined by averaging the signal over a large number of bins. In other embodiments, the background level is determined from the mean noise level, or the root-mean-square noise. In other cases, a typical noise value is chosen or a statistical value. In most cases, the noise is expected to follow a Poisson distribution.

In various embodiments, a threshold value is determined to discriminate true signals (peaks, bumps, single molecules) from noise. Care must be taken in choosing a threshold value such that the number of false positive signals from random noise is minimized while the number of true signals which are rejected is minimized. Methods for choosing a threshold value include determining a fixed value above the noise level and calculating a threshold value based on the distribution of the noise signal. In one embodiment, the threshold is set at a fixed number of standard deviations above the background level. Assuming a Poisson distribution of the noise, using this method one can estimate the number of false positive signals over the time course of the experiment.

In some embodiments, an analyzer or analyzer system utilized in the invention is capable of detecting an analyte, e.g., a biomarker at a level of less than 1 nanomolar, or 1 picomolar, or 1 femtomolar, or 1 attomolar, or 1 zeptomolar. In some embodiments, the analyzer or analyzer system is capable of detecting a change in concentration of the analyte, or of multiple analytes, e.g., a biomarker or biomarkers, from one sample to another sample of less than about 0.1, 1, 2, 5, 10, 20, 30, 40, 50, 60, or 80% when the biomarker is present at a concentration of less than 1 nanomolar, or 1 picomolar, or 1 femtomolar, or 1 attomolar, or 1 zeptomolar, in the samples, and when the size of each of the sample is less than about 100, 50, 40, 30, 20, 10, 5, 2, 1, 0.1, 0.01, 0.001, or 0.0001 μL. In some embodiments, the analyzer or analyzer system is capable of detecting a change in concentration of the analyte from a first sample to a second sample of less than about 20%, when the analyte is present at a concentration of less than about 1 picomolar, and when the size of each of the samples is less than about 50 μL. In some embodiments, the analyzer or analyzer system is capable of detecting a change in concentration of the analyte from a first sample to a second sample of less than about 20%, when the analyte is present at a concentration of less than about 100 femtomolar, and when the size of each of the samples is less than about 50 μL. In some embodiments, the analyzer or analyzer system is capable of detecting a change in concentration of the analyte from a first sample to a second sample of less than about 20%, when the analyte is present at a concentration of less than about 50 femtomolar, and when the size of each of the samples is less than about 50 μL. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 50 μL. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 5 μL. In some embodiments, the analyzer or analyzer system is capable of detecting a change in concentration of the analyte from a first sample to a second sample of less than about 20%, when the analyte is present at a concentration of less than about 1 femtomolar, and when the size of each of the samples is less than about 5 μL.

Single molecules of the analyte or analytes can be detected by detection of one or more label on one or more detectable species. In an exemplary embodiment of the invention, the extrinsic properties that render the single molecule of the detectable species detectable are provided by at least two labels. For example, the target single molecule is labeled with two or more labels and each label is distinct due to detected emission at one or more wavelengths that is distinguishable from the emission of the other label(s). In this example, the single molecule is distinguished from free label (or that adventitiously bound to an analyte or sample component) by the ratio of detected emission at two or more wavelengths. In another example, the single molecule is labeled with two or more labels and at least two of the labels emit at the same wavelength. In this example, single molecules are distinguished on the basis of the intensity of the detected fluorescence produced by emission from the two, three, or more labels attached to each single molecule.

In another embodiment, the dyes have the same or overlapping excitation spectra, but possess distinguishable emission spectra. Preferably dyes are chosen such that they possess substantially different emission spectra, preferably having emission maxima separated by greater than about 10 nm, more preferably having emission maxima separated by greater than about 25 nm, even more preferably separated by greater than about 50 nm. When it is desirable to differentiate between the two dyes using instrumental methods, a variety of filters and diffraction gratings allow the respective emission spectra to be independently detected. Instrumental discrimination can also be enhanced by selecting dyes with narrow bandwidths rather than broad bandwidths; however, such dyes must necessarily possess a high amplitude emission or be present in sufficient concentration that the loss of integrated signal strength is not detrimental to signal detection.

In various embodiments using more than one label, a second label may quench the fluorescence of a first label, resulting in a loss of fluorescent signal for doubly labeled single molecules. Examples of suitable fluorescing/quenching pairs include 5′ 6-FAM™/3′ Dabcyl, 5′ Oregon Green® 488-X NHS Ester/3′ Dabcyl, 5′ Texas Red®-X NHS Ester/3′ BlackHole Quencher™-1 (Biosearch Technologies, Novato, Calif.).

In another example, two labels may be used for fluorescence resonance energy transfer (“FRET”), which is a distance-dependent interaction between the excited states of two dye single molecules. In this case, excitation is transferred from the donor to the acceptor single molecule without emission of a photon from the donor. The donor and acceptor single molecules must be in close proximity (e.g., within about to about 100 Å). Suitable donor, acceptor pairs include fluorescein/tetramethylrhodamine, LAEDANS/fluorescein, EDANS/dabcyl, fluorescein/QSY7, (R. Haugland, “Molecular Probes,” Ninth edition, 2004) and many others known to one skilled in the art.

Single molecules may be labeled with more than one kind of label, such as a dye tag and a mass tag, to facilitate detection and/or discrimination. For example, an analyte may be labeled with two detectable species (e.g., two differentiatable antibodies), one that is unlabeled and acts as a mass or mass/charge tag, and another that has a dye tag. In some embodiments, the protein is distinguished from another protein of similar size that is bound only to an antibody with a dye tag by its slower velocity when, e.g., electrophoresis is used as the motive force (caused by the increased mass or mass/charge of the additional bound antibody).

### Data Processing

After collection of the single molecule detection data in the sample, in various embodiments, the data is compared to a reference set of data, acquired under the same experimental conditions, to determine if variations exist in the single molecule detection of the analyte which are characteristic of differences of analytical, diagnostic or prognostic value. In exemplary embodiments, differences between the experimental and reference data are indicative of a disease state, progress of a disease state, stage of a disease state or efficacy of a treatment modality for the disease state. A number of means of performing this comparison are of utility. In an exemplary embodiment, multivariate analysis is used.

Discrimination between data acquired from samples having subtle variations requires the use of robust and sensitive methods of analysis. These methods must model for the nonlinearities that can arise due to various causes as well as account for the day to day drifts in instrument settings. Sample handling errors, noise fringes, baseline shifts, batch to batch variations, the presence of nondiagnostic debris and all other factors that adversely affect discrimination are also preferably adequately accounted for and modeled. Lastly, for a method to prove robust it must distinguish between good and poor quality data, and exclude samples not representative of the reference set. The non-representative samples are referred to as outlier samples. An outlier sample is a sample that is statistically different from all other samples in the reference set. In the case of single molecule detection of species such as cytokines, an outlier data set can result from samples with less than an optimal number of copies of the single molecule, and/or specimens that are rich in nondiagnostic debris.

In various embodiments, the reference data set is representative of all expected variations in the data set for a selected single molecule. The data of all samples is then processed using methods such as, for example, classical methods of spectroscopic data analysis or multivariate analysis.

Multivariate analysis has been used to analyze biological samples. For example, Robinson, et al. in U.S. Pat. No. 4,975,581 (issued Dec. 4, 1990) describe a quantitative method to determine the similarities of a biological analyte in known biological fluids using multivariate analysis.

Principal Component Analysis (PCA) and discriminate analysis are employed to distinguish between normal and abnormal biological samples. See, Ge, et al., Applied Spectroscopy 49:432-436 (1995). Haaland, et al., in U.S. Pat. No. 5,596,992 (issued Jan. 28, 1997) teach the use of multivariate methods to detect differences between normal and malignant cell samples.

In the present invention, when multivariate analysis is used, the comparison of single molecule detection data can be carried out by a partial least squares (PLS) or principal component analysis (PCA) statistical method on data which can be preprocessed (i.e., smoothed and/or derivatized), or unsmoothed and underivatized. Preferably, comparisons using principle component regression (PCR) are carried out using PCA. A number of computer programs are available which carry out these statistical methods, including PCR-32® (from Bio-Rad, Cambridge, Mass., USA) and PLS-PLUS® and DISCRIMINATE° (from Galactic Industries, Salem, N.H., USA). Discussions of the underlying theory and calculations can be found in, for example, Haaland, et al., Anal. Chem. 60:1193-1202 (1988); Cahn, et al., Applied Spectroscopy, 42:865-872 (1988); and Martens, et al., MULTIVARIATE CALIBRATION, John Wiley and Sons, New York, N.Y. (1989). Both PCR and PLS use a library of data derived from single molecules of a reference single molecule sample to create a reference set, wherein each of the data sets are acquired under essentially identical conditions. The data analysis techniques consist of data compression (in the case of PCR, this step is known as PCA), and linear regression. Using a linear combination of factors or principal components, a reconstructed data set is derived. This reconstructed data set is compared with the data set of unknown specimens which serves as the basis for classification.

In certain aspects of the present invention the predicted scores generated for individual data sets are “averaged” over the data sets acquired for a particular species of detected single molecule. The “averaged” score from the individual data sets over the sample are “averaged” over the collection of samples. The method of “averaging” can consist of simply taking the arithmetic mean of the predicted scores or can rely on other statistical methods for determining population distributions known in the art. The methods include, for example, determining the median and determining the mean of the predicted score population. The extent to which a population is scattered on either side of the determined center is assessed by establishing a measure of dispersion such as, for example, the standard deviation, the interquartile range, the range and the mean deviation. Other methods, of use in practicing the present invention, for establishing both the “average” value for the population of prediction scores and the extent of population scatter will be apparent to those of skill in the art.

Prior to the analysis of unknown samples, another set of data of the same single molecule can be used to validate and optimize the reference. This second set of data enhance the prediction accuracy of the PCR or PLS model by determining the rank of the model. The optimal rank is determined from a range of ranks by comparing the PCR or PLS predictions with known diagnoses. Increasing or decreasing the rank from what was determined optimal can adversely affect the PLS or PCR predictions. For example, as the rank is gradually decreased from optimal to suboptimal, PCR or PLS would account for less and less variations in the reference spectra. In contrast, a gradual increase in the rank beyond what was determined optimal would cause the PCR or PLS methodologies to model random variation rather than significant information in the reference spectra.

Generally, the more single molecule detection data a reference set includes, the better is the model, and the better are the chances to account for batch to batch variations, baseline shifts and the nonlinearities that can arise due to instrument drifts and changes in the refractive index. Errors due to poor sample handling and preparation, sample impurities, and operator mistakes can also be accounted for so long as the reference data render a true representation of the unknown samples.

Another advantage to using PCR and PLS analysis is that these methods measure the data noise level of unknown samples relative to the reference data. Biological samples are subject to numerous sources of perturbations. Some of these perturbations drastically affect the quality of data, and adversely influence the results of a “diagnosis”. Consequently, it is preferable to distinguish between data that conform with the reference data, and those that do not (e.g., the outlier samples). The F-ratio is a powerful tool in detecting conformity or a lack of fit of a data set (sample) to the reference data. In general F-ratios considerably greater than those of the reference indicate “lack of fit” and should be excluded from the analysis. The ability to exclude outlier samples adds to the robustness and reliability of PCR and PLS as it avoids the creation of a “diagnosis” from inferior and corrupted data. F-ratios can be calculated by the methods described in Haaland, et al., Anal. Chem. 60:1193-1202 (1988), and Cahn, et al, Applied Spectroscopy 42:865-872 (1988).

When discriminating between single molecule data from different samples, the biological materials no longer have known concentrations of constituents. As a result, in exemplary embodiments, the reference data determines the range of variation allowed for a sample to be classified as a member of that reference, and should also include preprocessing algorithms to account for diversities in sample make up.

Other data processing algorithms are of use in the present invention. For example in one exemplary embodiment of the present invention, diagnostically valuable cytokines (or other markers) may be first identified using a statistically weighted difference between control individuals and diseased patients, calculated as D−NσD*σ_{N} where D is the median concentration of a cytokine in patients diagnosed as having a particular disease, N is the median of the control individuals, (D* is the standard deviation of D and σ_{N} is the standard deviation of N. The larger the magnitude, the greater the statistical difference between the diseased and normal populations.

According to one embodiment of the invention, cytokines resulting in a statistically weighted difference between control individuals and diseased patients of greater than 0.2, 0.5, 1, 1.5, 2, 2.5 or 3 are identified as diagnostically valuable markers.

As will be appreciated by those of skill, the patient him/herself may be the “control”. For example, if the assay is part of monitoring a course of treatment or of disease progression, data acquired from the patient at the start of the course of treatment or upon discovery or diagnosis of the disease can serve as a reference data set for assessing changes in the disease markers due to treatment or disease progression. Other embodiments in which data from the patient providing a sample for an assay of the invention serves as a reference data set or baseline data set will be apparent to those of skill in the art.

Another method of statistical analysis of use in the methods of the invention for determining the efficacy of particular candidate analytes, such as particular cytokine(s), for acting as diagnostic marker(s) is Receiver Operating Characteristic (ROC) curve analysis. An ROC curve is a graphical approach to looking at the effect of a cut-off criterion (e.g., a cut-off value for a diagnostic indicator such as an assay signal or the level of an analyte) on the ability of a diagnostic to correctly identify positive and negative samples or subjects. In an exemplary analysis, one axis of the ROC curve is the true positive rate (TPR, the probability that a true positive sample/subject will be correctly identified as positive) or, alternatively, the false negative rate (FNR=1-TPR, the probability that a true positive sample/subject will be incorrectly identified as a negative). The other axis is the true negative rate (TNR, the probability that a true negative sample will be correctly identified as a negative) or, alternatively, the false positive rate (FPR=1-TNR, the probability that a true negative sample will be incorrectly identified as positive). The ROC curve is generated using assay results for a population of samples/subjects by varying the diagnostic cut-off value used to identify samples/subjects as positive or negative and plotting calculated values of TPR (or FNR) and TNR (or FPR) for each cut-off value. The area under the curve (referred to herein as the ROC area) is one indication of the ability of the diagnostic to separate positive and negative samples/subjects.

One of skill in the art of diagnostic assays and statistical analysis of data, given the teaching and guidance provided herein, will be able to select without undue burden appropriate cut-off values, lines, ratios, zones etc. for best meeting the needs (e.g., sensitivity and specificity) for a particular application. A variety of statistical tools, such as, for example, receiver operating characteristic (ROC) curves, are available for evaluating the effect of adjustments to cut-offs on assay performance (e.g., predicted true positive fraction, false positive fraction, true negative fraction and false negative fraction). Alternatively, statistical analysis of patient populations can allow conversion of specific analyte values into probabilities that the patient has or does not have a disease. For background on the selection and analysis of populations of individuals so as to determine reference ranges see Boyd J. C. “Reference Limits in the Clinical Laboratory” in Professional Practice in Clinical Chemistry: A Companion Text; D. R. Dufour Ed., 1999, Washington D.C.: American Assoc. CLIN. CHEM., Chapter 2, pp. 2-1 to 2-7. For background on the selection of decision limits (i.e., cut-offs) or the calculation, from test results, of disease likelihood see Boyd J. C. “Statistical Aids for Test Interpretation” in Professional Practice in Clinical Chemistry: A Companion Text; D. R. Dufour Ed., 1999, Washington D.C.: American Assoc. CLIN. CHEM., Chapter 3, pp. 3-1 to 3-11.

Given the teachings of the present invention, a skilled artisan will also recognize that the choice of first marker (e.g., a first cytokine) and one or more additional marker (e.g. a second cytokine) may transpose correlation plot axes and consequently the criteria for determining whether measured marker levels of a patient's samples falling above or below particular cut-off ratios, lines and/or profiles is indicative of a disease state and will be able to adjust the analysis accordingly.

The present invention also provides a data set acquired from single molecule analysis of an analyte. In an exemplary embodiment, the data set includes at least one data point corresponding to a signal from an analyte collected from an assay for a single molecule of said analyte, said assay having a limit of detection of less than 0.5 pg/mL and a dynamic range of at least 2.5 log.

In a further aspect, the invention provides business methods. In one embodiment, the invention provides a method of doing business comprising use by an entity of a method of the invention to obtain a result for an assay of a sample, reporting said result, and payment to the entity for the reporting of the result. In some embodiments, the entity is a Clinical Laboratory Improvement Amendments (CLIA) laboratory. In some embodiments, the entity is a laboratory that is not a CLIA laboratory. The sample may be any type of sample capable of being analyzed by the single particle detector. In some embodiments, the sample is from an individual. The individual may be any type of individual as described herein. In some embodiments, the individual is a patient (e.g., animal, e.g., human) for which screening, diagnosis, prognosis, monitoring and/or determination of method of treatment is desired. In some embodiments, the individual is an individual (e.g. animal, e.g. human) who is participating in a clinical trial or in pre-clinical trial research. In some embodiments, the sample is from an individual who is part of a research project, e.g., biomedical research, agricultural research, industrial research, educational research, bioterrorism research, and the like. In some embodiments, payment may be by the individual receiving the report of the result, e.g., a health care professional and/or the individual from whom the sample was taken, to the entity performing the analysis, e.g., a CLIA laboratory, or it may be by the individual from whom the sample was taken to the individual receiving the report from the entity performing the analysis, or to the entity itself, or both, or some combination thereof. In another embodiment, the invention provides a method of doing business, comprising use of a detector with two interrogation spaces that is capable of detecting single particles by a health-care provider to obtain a result for an assay of a sample from an individual, reporting said result to the individual or their representative; and payment by the individual for said reporting of the result.

### Kits

The present invention also provides kits for performing an analysis of the invention. In an exemplary embodiment, the kit includes one or more capture species, detectable species, and magnetic particle. The kit also includes a reagent or reagents of use in performing the method of the invention. Also provided are instructions for performing the assay of the invention. An exemplary kit and its use in detecting IL-6 is set forth in Example 2.

The following examples are provided to illustrate certain embodiments of the invention and are not limiting of these or any other embodiments of the invention.

### EXAMPLES

### Example 1

### Materials and Methods

### Materials

Antibodies and analytes (recombinant) were obtained from R&D Systems (Minneapolis, Minn.). Manufacturer recommendations were followed for matched antibody pairs. Fluorescent dyes and biotin succinimidyl ester, used to label antibodies, were obtained from Invitrogen (Carlsbad, Calif.). Rat, dog, and monkey cTnI were purified from natural sources and obtained from Hytest (Sweden). Human lithium citrate plasma specimens were purchased from Interstate blood bank (St. Louis, Mo.). Streptavidin coated paramagnetic microparticles (MPs) were obtained from Invitrogen (MyOne, #650-01) Antibodies were labeled with fluorescent dye (detection antibody, usually polyclonal) and biotin (capture antibody, usually monoclonal) using manufacturers recommendations. MPs were coated with biotinylated antibody under saturation conditions (following manufacturer's recommendations), washed and stored in assay buffer. Assay buffer consisted of 1% BSA, Tris buffered saline, pH 7.4, 0.05% TritonX-100 and heterophile/HAMA antibody blocking reagents (purchased from Scantibodies Laboratories, Roche Life Sciences) and used per manufacturer's recommendations.

### Erenna Immunoassays

Unless stated otherwise, the typical Erenna immunoassay was performed as follows. Samples or standards (e.g. 50 μL-100 μL) were diluted with assay buffer containing capture antibody coated MPs (e.g. 150 μL) and incubated in a 96 well plate for one to two hours at 25° C. with shaking. All plasma or serum samples were tested neat without pre-treatment. MPs were separated using a magnetic bed (Ambion, Tex.). Supernatant was removed, MPs washed once and then 20 μL of detection antibody (50-500 μg/mL diluted in assay buffer) was added and incubated for 60 minutes at 25° C. with shaking. The MPs were again magnetically separated and washed six times using Tris buffered saline plus 0.05% Triton X-100. After removal of residual wash buffer, 20 μL of elution buffer (4M urea) was added. This reagent disrupted antibody-analyte interactions and resulted in the release of detection antibody from the MPs. The solution in each 96-well was then transferred to a 384-well filter plate (0.2 micron, AcroPrep, East Hills N.Y.) and centrifuged at 3,000 RPM for three minutes to separate detection antibody in elution buffer from MPs. The eluted and filtered material in the 384-well plate was then placed into the Erenna Immunoassay System (Singulex, Inc.).

### Erenna Immunoassay System

The Erenna Immunoassay System is based upon single molecule counting technology. Liquid is sipped from each well in the 384 well plate and pumped through a capillary flow cell with (100 micro-meter diameter). The liquid passes through an interrogation space within the capillary. As depicted in FIG. 1, light generated from a laser is directed via a dichroic mirror and a confocal microscope lens into the interrogation space. As dye-labeled antibodies pass through this space, they emit fluorescent light which is measured via the confocal microscope lens and a photon detector. The output from the detector is a train of pulses with each pulse representing one photon that was detected. These pulses are sent to counting electronics where the pulses are counted in 1 milli-second bins. The 4.5 plus log dynamic reporting range is obtained by using a combination of output signals. In the first, background level is determined and based on this level a 5 standard deviation threshold above background is created. Only flashes of light that are greater than this threshold are counted. These individual peaks (not signal intensity) are summed over either a one minute interval or until 1,000 peaks are obtained. The final signal is a sum of all such measured events and is termed detected events (DE). The second output is termed event photons (EP) and is the sum all the photons counted in all the detected events. This measure is used at higher concentrations when there is a significant probability that two molecules will pass through the detector in the same 1 ms counting bin. At the highest concentrations of analyte, EP events begin to saturate and total photons (TP), the sum of all photon events, is used. DE, EP and TP signal are used to generate a weighted four-parameter logistics curve fit for each signal type. To estimate the concentration of an unknown, the DE, EP and TP signals are interpolated off each of the standard curves to obtain three separate estimates of concentration. These three concentrations are combined using a weighted average based upon the slopes of the standard curves. This provides a >4.5 log linear reporting range.

### Results

This section presents representative data from a series of different experiments using different analytes with the intent of providing an overall perspective of the performance of the Erenna Immunoassay System.

### Reporting Range, Linearity, Accuracy and Reproducibility

An example of typical signal data generated with a human IL-17 assay is presented in Table 1. The DE, EP and TP signal that are used to construct the standard curve are presented in a bold font. To determine accuracy of the curve fit, the signal values in Table 1 were back interpolated using the curve fit algorithm and the results are presented as measured vs. expected pg/mL.

TABLE 1
Signal, back interpolated values and recovery from a human IL-17 Erenna Assay standard curve.

| IL-17 | # Detected Events/min | | | # Photon Events/min | | | # Total Photons/min | | | Measured IL-17 pg/mL | | | |
|---|---|---|---|---|---|---|---|---|---|---|---|---|---|
| pg/mL | Mean | SD | CV | Mean | SD | CV | MEAN | SD | CV | MEAN | SD | CV | Bias |
| 1,000 | 8,920 | 2,268 | 25% | 13,879,788 | 157,439 | 1% | 159,161,873 | 2,366,288 | 1% | 843 | 23.00 | 3% | 84% |
| 500 | 10,538 | 78 | 1% | 10,910,741 | 285,636 | 3% | 88,854,604 | 233,442 | 0% | 468 | 1.23 | 0% | 94% |
| 250 | 10,185 | 171 | 2% | 7,515,849 | 104,214 | 1% | 46,946,733 | 3,717,269 | 8% | 238 | 20.03 | 8% | 95% |
| 125 | 10,250 | 217 | 2% | 5,237,108 | 363,085 | 7% | 25,753,075 | 1,564,066 | 6% | 119 | 9.74 | 8% | 96% |
| 63 | 10,005 | 45 | 0% | 3,485,980 | 178,040 | 5% | 14,781,797 | 870,996 | 6% | 59 | 4.79 | 8% | 94% |
| 31 | 9,639 | 182 | 2% | 2,406,459 | 91,291 | 4% | 9,325,716 | 326,061 | 3% | 32 | 1.59 | 5% | 102% |
| 16 | 8,507 | 103 | 1% | 1,564,471 | 103,417 | 7% | 6,690,690 | 176,886 | 3% | 18 | 1.39 | 8% | 116% |
| 7.8 | 6,428 | 211 | 3% | 915,104 | 47,403 | 5% | 5,043,196 | 52,626 | 1% | 9.6 | 0.57 | 6% | 123% |
| 3.9 | 3,999 | 54 | 1% | 459,594 | 16,948 | 4% | 4,077,945 | 30,411 | 1% | 4.2 | 0.14 | 3% | 108% |
| 1.95 | 2,111 | 192 | 9% | 212,996 | 20,496 | 10% | 3,634,021 | 33,976 | 1% | 1.73 | 0.19 | 11% | 88% |
| 0.98 | 1,189 | 33 | 3% | 113,214 | 457 | 0% | 3,414,771 | 10,274 | 0% | 0.88 | 0.02 | 3% | 90% |
| 0.24 | 342 | 14 | 4% | 31,199 | 628 | 2% | 3,256,437 | 6,346 | 0% | 0.22 | 0.01 | 5% | 92% |
| 0.12 | 211 | 8 | 4% | 17,901 | 870 | 5% | 3,236,763 | 24,896 | 1% | 0.13 | 0.01 | 5% | 103% |
| 0.06 | 125 | 15 | 12% | 10,823 | 1,176 | 11% | 3,203,629 | 3,476 | 0% | 0.06 | 0.01 | 19% | 99% |
| 0 | 64 | 12 | 19% | 6,013 | 2,235 | 37% | 3,211,483 | 8,892 | 0% | ND | — | — | |
| | | | | | | | | | | | | Average | 99% |

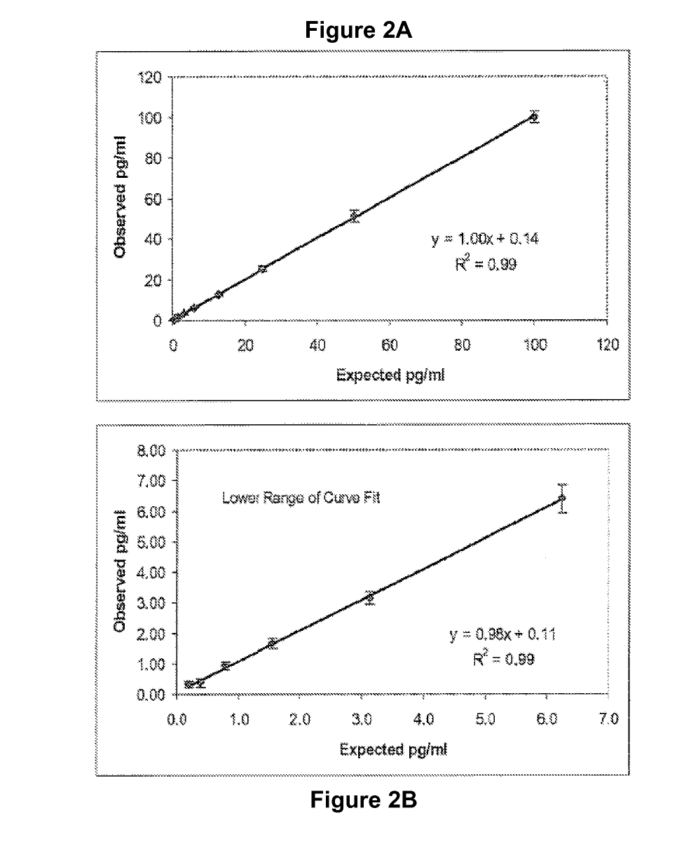

Average bias (measured/expected) was 99% (range 84-123%) and a linear response (R²=0.99; y=0.86+5.4) was observed from 60 fg/mL to 1 ng/mL, representing an accurate curve fit over a 4.3 log reporting range. Accuracy of quantification in biological samples was determined by measuring spike recovery of analyte (IL-17, IL-6 and human cardiac troponin-I, cTnI) into panels of human plasma (5 and 50 spike pg/mL). Average % recoveries were between 90% and 110% for at both concentrations (data not shown). The accuracy of the curve fit algorithm using the Erenna cTnI immunoassay over eight consecutive runs (over 6 days using one lot of reagents and freshly prepared calibrators each day) and back interpolating the standard curves are presented in FIG. 2. In these experiments, the highest concentration standard used was 100 pg/mL. A linear (R²=0.99) response was observed at both the high and low ends of the standard curve shown. The CV for the 8 assay runs for back interpolated determinations of calibrators was <10% for all values>0.78 pg/mL. The CV was 16% and 23% for the 0.39 and 0.2 pg/mL values, respectively (Table 2). Inter-assay and intra-assay precision studies, using plasma or sera spiked with known amounts of analyte were performed. As an example of results obtained with these experiments, the human IL-17 assay yielded intra-assay (replicates of 4) CVs ranging from 3-8% (IL-17>0.4 pg/mL) and 10% (0.2 pg/mL) as well as inter-assay (6 assay runs) CVs ranging from 3-9% (IL-17>0.4 pg/mL) and 11% (0.2 pg/mL).

TABLE 2
Reproducibility of the human cTnI assay calibration system over 8 assay runs (6 days).

| cTnI | | | | | | | | | MEAN | | |
|---|---|---|---|---|---|---|---|---|---|---|---|
| pg/mL | RUN 1 | RUN 2 | RUN 3 | RUN 4 | RUN 5 | RUN 6 | RUN 7 | RUN 8 | pg/mL | SD | CV |
| 100 | 101 | 101 | 104 | 96 | 97 | 99 | 99 | 98 | 100 | 3 | 3% |
| 50 | 46 | 55 | 50 | 53 | 51 | 51 | 51 | 50 | 51 | 3 | 6% |
| 25 | 26 | 24 | 24 | 26 | 26 | 27 | 24 | 25 | 25 | 1 | 4% |
| 12.5 | 11.4 | 12.3 | 12.5 | 12.7 | 12.7 | 12.2 | 12.6 | 12.6 | 12.4 | 0.5 | 4% |
| 6.3 | 6.6 | 6.1 | 6.2 | 6.2 | 6.5 | 6.5 | 6.6 | 6.3 | 6.4 | 0.2 | 3% |
| 3.1 | 3.0 | 3.1 | 3.2 | 2.9 | 3.4 | 3.2 | 3.2 | 3.1 | 3.2 | 0.2 | 5% |
| 1.6 | 1.7 | 1.7 | 1.6 | 1.6 | 1.8 | 1.8 | 1.4 | 1.5 | 1.7 | 0.1 | 8% |
| 0.78 | 0.93 | 0.91 | 1.16 | 0.94 | 0.66 | 0.87 | 0.97 | 0.74 | 0.92 | 0.15 | 16% |
| 0.39 | 0.38 | 0.49 | 0.30 | 0.40 | 0.25 | 0.44 | 0.32 | 0.49 | 0.37 | 0.08 | 23% |
| 0.2 | 0.12 | 0.50 | 0.19 | 0.29 | 0.34 | 0.44 | 0.34 | 0.52 | 0.32 | 0.13 | 42% |
| 0.1 | 0.00 | 0.03 | 1.02 | 0.26 | 0.11 | 0.05 | 0.05 | 0.23 | 0.22 | 0.36 | 168% |

### Sensitivity

Analytical sensitivity or limit of detection (LoD; defined as two standard deviations of the signal from the zero analyte “background” divided by the slope of the linear portion of DE signal; which represents the lowest level of analyte concentration that can be differentiated from the background signal with 95% confidence) for the Erenna immunoassay system varied from analyte to analyte and was dependent upon the volume of sample (standard) used. Table 3 depicts the LoD for 10 different Erenna human immunoassays using two different sample volumes for each assay.

TABLE 3
The effect of sample volume on Erenna System immunoassay sensitivity
(LoD)

| | Volume | LoD | Volume | LoD | |
|---|---|---|---|---|---|
| Analyte | (μL) | (pg/mL) | (μL) | (pg/mL) | Proportionality |
| MCP-1 | 200 | 0.03 | 20 | 0.25 | 120% |
| RANTES | 100 | 0.05 | 20 | 0.3 | 120% |
| VEGF | 100 | 0.12 | 20 | 0.55 | 109% |
| IL-8 | 50 | 0.12 | 20 | 0.3 | 100% |
| IL-1a | 200 | 0.01 | 20 | 0.1 | 100% |
| IL-7 | 200 | 0.02 | 10 | 0.3 | 133% |
| Il-6 | 100 | 0.01 | 10 | 0.13 | 78% |
| TNF-a | 200 | 0.02 | 10 | 0.4 | 100% |
| Il-1B | 150 | 0.02 | 10 | 0.3 | 100% |
| cTnI | 100 | 0.11 | 10 | 1.2 | 92% |

Larger sample volumes consistently resulted in lower LoDs and enhanced sensitivity. For example, sample volumes>50 μL resulted in LoDs ranging from 0.01 pg/mL (human IL-6) to 0.12 pg/mL (human VEGF). As sample volumes decreased, LoDs increased in a proportional manner (average proportional relationship 105%). We have found that the volumetric ratios of assay buffer to sample (plasma) need to be varied, in an assay specific manner, to achieve optimal sensitivity, recovery of analyte spiked into sample and dilutional linearity. The assay volumes presented in Table 3 represent such optimization. All of the assays were performed in a two-step manner with 1-2 hours of capture and 1 hour of detection.

Experiments were performed to understand the impact of assay incubation times on assay sensitivity. The goal of these experiments was to understand the potential applicability of using the Erenna System in a rapid testing environment. To achieve this, incubation steps were combined into a single step cTnI assay was performed using 50 μL of sample (plus 150 μL assay buffer) and simultaneous capture and detection (10 μg/well MPs and 100 ng/mL detection antibody) reactions. As incubation times, increased assay sensitivity improved. This was due to an improvement in slope response as a function of time (15 min, 64 slope; 30 min, 90 slope; 60 min, 102 slope; 120 min, 152 slope) while assay background, (DE signal of the zero analyte calibrator) remained constant over time. Of note, even with 15 minute incubation a LoD of 0.61 pg/mL was achieved, using the NIST reference material (data not shown).

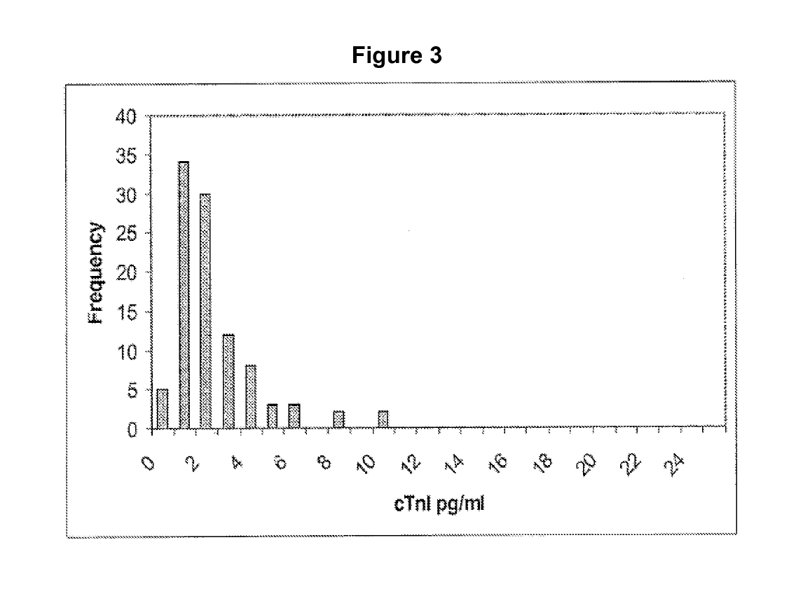

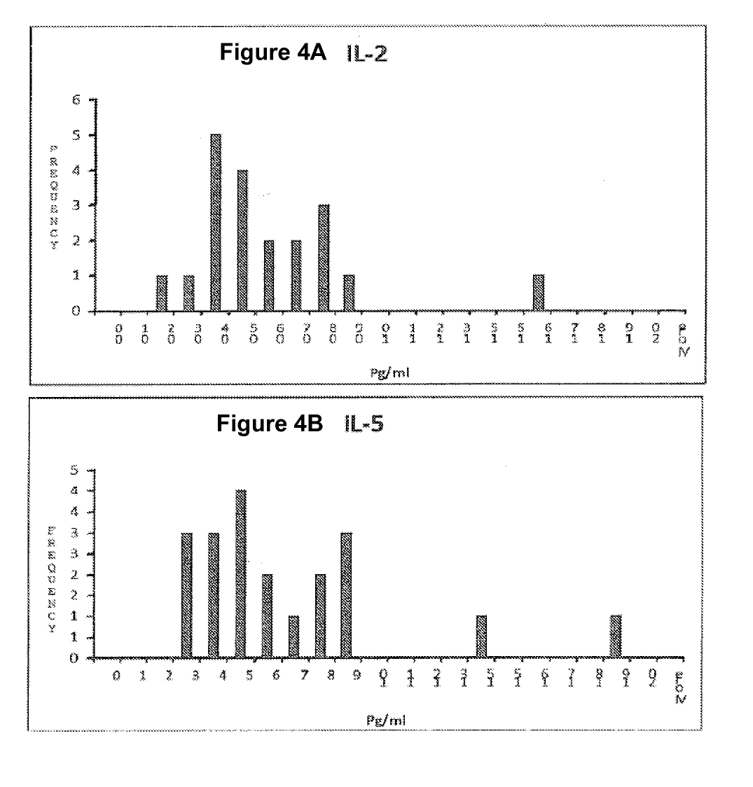

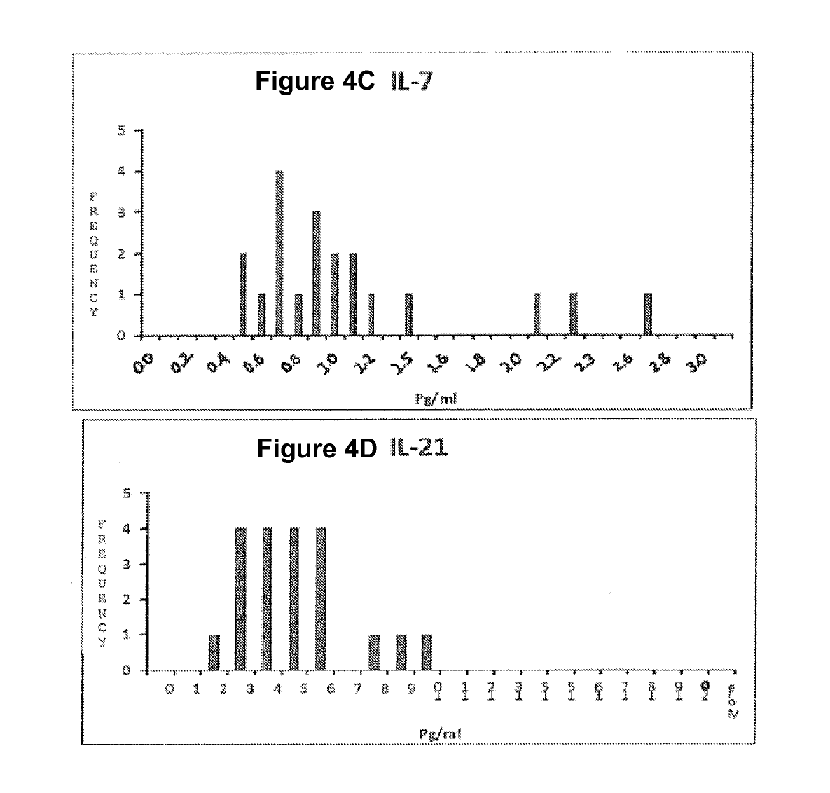

The sensitivity of the human cTnI assay was employed to define the range of cTnI in human plasma obtained from 100 human blood donors (50 μL sample size). The results are presented in FIG. 3. The values ranged from <2 pg/mL to 39 pg/mL with an average value of 2.19+/−4.1 pg/mL. Three plasma had values<0.2 pg/mL which was the LoD. The value for the 99^{th} percentile was 9 pg/mL. If the one apparent outlier of 39 pg/mL was removed from the data set the mean for the population was 1.83+/−1.9 pg/mL with the 99^{th} percentile at 8 pg/mL.

### Example 2

### Materials and Methods

### Materials

| Item # | Description | Shipping Conditions |
|---|---|---|
| 1. | Human IL-6 Standard Diluent | With cold pack |
| 2. | Human IL-6 Capture Reagent | With cold pack |
| 3. | Human IL-6 Detection Reagent | With cold pack |
| 4. | Erenna^{tm}Human IL-6 Immunoassay Kit | N/A |
| | Instructions | |
| 5. | Human IL-6 Standard | On Dry Ice |
| | (frozen, shipped in separate box) | |
| 6. | 10X Wash Buffer | With cold pack |
| 7. | Elution Buffer | With cold pack |

### Additional Supplies

| | | | Component | | |
|---|---|---|---|---|---|
| Item | | Mfr | Part | | Packaging |
| ## | Description | Supplier | Numbers | Product Uses | Detail |
| 1. | Erenna ™ | Singulex | 02-0111- | Systems | 1 L (10 L |
| | 10X Systems | | 00, 02- | (Analysis) Buffer, | mixed) |
| | Buffer | | 0111-01 | fluid used to run | 2 L (20 L |
| | | | | Erenna System | mixed) |
| 2. | Reservoirs for | VWR | 80092-466 | Transfer of reagents | 10/pkg |
| | 12-Channel | | | | |
| | Pipetters | | | | |
| 3. | 96-Well V- | Axygen | P-96-450V- | Additional assay | 10 |
| | Bottom PP | | C or P-96- | plate, dilutions | plates/unit |
| | Plate, 500 μL | | 450V-C-S | | 5 |
| | | | | | units/case |
| 4. | 96-Well Deep | Axygen | P-2ML-SQ- | Prepare standard | Variable |
| | Well PP Plate | | C, | curves (choose size) | |
| | (2.2 mL, 1.64 mL | | P-DW-20-C | | |
| | or 1.09 mL) | | or | | |
| | | | P-DW-11-C | | |
| 5. | 384-Well | Nunc | 264573 | Receiver/analysis | 20/pk or |
| | Round Bottom | | | plate | 120/cs |
| | PP, 120 μL | | | | |
| 6. | AcroPrep ™ | Pall | 5070 | Remove MPs from | 10/pkg |
| | 384-Filter | | | assay | |
| | Plates, 100 μL, | | | | |
| | for sample | | | | |
| | preparation | | | | |
| | and detection | | | | |
| 7. | Advanced | Nunc | 235306 | Permanent seal for | 100 units/pk |
| | Pierceable | | | analysis plate, used | 100 pks/cs |
| | Sealer, | | | prior to Erenna run | |
| | Polyethylene | | | | |
| 8. | AxySeal- | Axygen | PCR-SP | Sealing plates | 100 films/ |
| | PCRSP Plate | | | during | case |
| | sealing film | | | incubation/mix/store | |
| | series | | | | |

### Magnetic Particles

| Item | | Mfr | Component | | Pkg |
|---|---|---|---|---|---|
| ## | Description | Supplier | Part ## | Product Uses | Detail |
| 1. | Dynal | Dynal ™ | 120.27 | Rare Earth | 1 |
| | MPC ®-96S | | | Magnet, | plate |
| | | | | capture MP | |
| | | | | during wash | |
| 2. | Microplate | — | — | Wash MP | — |
| | Wash Station | | | following | |
| | | | | capture on | |
| | | | | magnet | |
| 3. | Centrifuge | — | — | Remove MP | 1 |
| | w/Plate | | | via filter | |
| | Rotor | | | plate | |
| | | | | ~1,200 × g | |
| 4. | Centrifuge | Pall | 5225 | Creates fit b/ | 2/pkg |
| | Adapter | | | n 384-well | |
| | Collar | | | filter plate | |
| | | | | 384-well | |
| | | | | assay plate | |
| 5. | Vacuum | Welch | 2511B-01 | Degassing | 1 |
| | Pump | | | systems buffer | |
| 6. | Microplate | Boekel | # 130000 | Incubating | 1 |
| | Incubator/ | Scientific | The | plate | |
| | Shaker | | Jitterbug ™ | | |
| 7. | Plate Seal | VWR | 60941-118 | Secures plate | 1 |
| | Roller, VWR | | | seal permanent | |
| | Plate Roller, | | | plate seal | |
| | Film + Foil | | | | |
| | CS1# | | | | |

### Procedure

Capture Reagent (100 μL) followed by 75 μL of Standards/Samples was added to each well of 96-well polypropylene plate. The plate was covered and incubated/shaken for two hours at room temperature. The cover was removed and the plate set onto a magnet for a time sufficient for the MP to settle/amass. The supernatant was removed. With the plate on the magnet, Wash Buffer (250 μL) was added. After 2 minutes, the buffer was removed. The plate was removed from the magnet and IL-6 Detection Reagent (20 μL) was added. The plate was pulse centrifuged at 120×g, then covered and incubated/shaken for 1 hour at room temperature. The plate was set on to a magnet for about 2 minutes while the MP's amassed. The supernatant was removed. Wash buffer (250 μL) was added and removed (3×) with MP magnetically amassed near the magnet. The particles were incubated for 2 minutes with each Wash Buffer application before aspirating the buffer between each cycle. The magnet was removed and Wash Buffer (250 μL) was added and the plate was shaken for 10 seconds to re-suspend MP. The entire contents of the plate were transferred to a new 96-well plate. Following the transfer, the plate was set on a magnet, and after 2 minutes, the supernatant was removed. The plate was removed from the magnet and Wash Buffer (250 μL) was added. The plate was shaken for 10 seconds. The washing steps were repeated. The plate was removed from the magnet and add Elution Buffer (20 μL) was added to each well. The plate was pulse centrifuged at 120×g. The plate was covered and incubated/shaken at room temperature for 30 minutes. A filter plate was set over a 384 well plate and the contents of the 96-well plate were transferred to the 384 well plate. The filter plate combo was covered and centrifuged for 1 minute at 1,200×g. The top filter plate was removed and discarded. The top filter plate was removed and the 384 well plate was covered with a pierceable plate seal cover and the assay mixtures were transferred to an optical reader (e.g., Erenna System, Singulex).

### Results

The following is an exemplary curve standard IL-6 curve.

| IL-6 | mean | | | | | | | | % | Est [C] | | |
|---|---|---|---|---|---|---|---|---|---|---|---|---|
| [pg/ml] | DE's | StDev | % CV | mean EP's | StDev | % CV | mean TP's | StDev | CV | mean | Stdev | % CV |
| 37 | 9,828 | 57 | 1 | 2,728,786 | 93615 | 3 | 9,688,040 | 843,592 | 9 | 37.9 | 1.7 | 4 |
| 18.5 | 7,462 | 200 | 3 | 1,442,595 | 85859 | 6 | 6,603,869 | 178,146 | 3 | 17.4 | 1.0 | 6 |
| 9.25 | 5,350 | 379 | 7 | 856,045 | 36887 | 4 | 5,889,034 | 109,781 | 2 | 9.9 | 0.7 | 7 |
| 4.63 | 3,016 | 249 | 8 | 420,548 | 36860 | 9 | 5,271,331 | 103,553 | 2 | 4.3 | 0.7 | 17 |
| 2.31 | 1,587 | 57 | 4 | 206,927 | 8213 | 4 | 4,961,905 | 1,033 | 0 | 2.3 | 0.1 | 4 |
| 1.16 | 922 | 30 | 3 | 118,740 | 5344 | 5 | 5,050,734 | 253,349 | 5 | 1.1 | 0.22 | 20 |
| 0.58 | 507 | 44 | 9 | 61,654 | 6614 | 11 | 4,903,511 | 49,051 | 1 | 0.58 | 0.07 | 12 |
| 0.29 | 310 | 30 | 10 | 37,029 | 5315 | 14 | 4,852,156 | 48,862 | 1 | 0.28 | 0.04 | 15 |
| 0.14 | 209 | 7 | 3 | 25,680 | 6027 | 23 | 4,798,611 | 18,735 | 0 | 0.14 | 0.01 | 8 |
| 0.07 | 178 | 14 | 8 | 18,455 | 1217 | 7 | 4,826,107 | 127,787 | 3 | 0.09 | 0.02 | 25 |
| 0.036 | 148 | 12 | 8 | 16,506 | 1485 | 9 | 4,803,060 | 29,556 | 1 | 0.06 | | |
| 0 | 108 | 8 | 7 | 11,956 | 363 | 3 | 4,787,510 | 23,021 | 0 | ND | — | — |

The present invention provides substantially novel methods for detecting chemical differences between cell types and for distinguishing between normal and diseased cell samples. While specific examples have been provided, the above description is illustrative and not restrictive. Many variations of the disclosed methods will be apparent to those of skill in the art upon review of this specification. The scope of the invention should, therefore, be determined not with the reference to the above description, but instead should be determined with reference to the appended claims along with their full scope of equivalents.

It will be apparent to one of skill in the art that the above described techniques, particularly the detection of single molecules of diagnostically relevant species, will have application to other diagnostic species besides those exemplified herein. The enumerated techniques set forth herein are by way of example and are not intended to limit the scope of the invention to use with the explicitly diagnostic species.

All publications, patents and patent applications mentioned in this specification are herein incorporated by reference into the specification to the same extent as if each individual publication, patent or patent application was specifically and individually indicated to be incorporated herein by reference in their entirety for all purposes.

1-40. (canceled)

41. A method of performing an assay detecting a single molecule of a detectable antibody correlating to a single molecule of an analyte to which said detectable antibody specifically binds, said method comprising:
(a) in a first vessel,
(i) forming a hapten-antibody complex between said analyte and a capture antibody specifically binding said analyte, wherein said capture antibody comprises biotin and is immobilized on a magnetic particle comprising streptavidin through interaction between said biotin and said streptavidin; and
(ii) following (i), contacting said complex with said detectable antibody which specifically binds to said analyte thereby forming an immobilized complex of said detectable antibody;
(b) transferring said immobilized complex to a second vessel free of said detectable antibody;
(c) eluting said detectable antibody from said immobilized complex; and
(d) detecting said single molecule of said detectable antibody.

42. The method of claim 41 wherein, prior to (a)(i), said single molecule of said analyte is removed from a middle layer of a three-layered plasma sample comprising an upper lipid layer, said middle layer comprising said single molecule of said analyte, and a lower fibrin clot layer; said single molecule of said analyte removed in an aliquot from said middle layer of said sample.

43. The method of claim 41 wherein said biotin is bound to said capture antibody at a site which is a member selected from an amine moiety and a hinge saccharyl moiety.

44. A method of performing an assay of a sample detecting a single molecule of a detectable species correlating to a single molecule of an analyte which is a member selected from a cytokine and a growth factor, to which said detectable species specifically binds, said assay capable of detecting said detectable species in an amount of less than or equal to about 5 pg/mL, and said assay having a dynamic range of at least about 2 log; said method comprising:
(a) in a first vessel,
(i) forming a complex between said analyte and a capture species specifically binding said analyte, wherein said capture species comprises biotin and is immobilized on a magnetic particle comprising streptavidin through interaction between said biotin and said streptavidin; and
(ii) following (i), contacting said complex with said detectable species which specifically binds to said analyte thereby forming an immobilized complex of said detectable species;
(b) detecting said single molecule of said detectable species.

45. The method of claim 44 further comprising, prior to (b),
(c) transferring said immobilized complex to a second vessel free of said detectable species.

46. The method of claim 44 further comprising, prior to step (b),
(d) eluting said detectable species from said immobilized complex.

47. The method of claim 44 wherein said assay is configured to allow detection of said analyte in a range correlating with a clinically healthy, non-diseased state of said subject.

48. A method of determining a member selected from diagnosis, prognosis, state of treatment and method of treatment based on a result of a single molecule assay, said method comprising:
(a) in a first vessel,
(i) forming a complex between said analyte and a capture species specifically binding said analyte, wherein said capture species is immobilized on a magnetic particle; and
(ii) following (i), contacting said complex with said detectable species which specifically binds to said analyte thereby forming an immobilized complex of said detectable species;
(b) transferring said immobilized complex to a second vessel free of said detectable species;
(c) eluting said detectable species from said immobilized complex;
(d) detecting said single molecule of said detectable species;
(e) quantifying a plurality of said single molecule, thereby determining a concentration of said single molecule; and
(f) correlating said concentration of said single molecule with a member selected from said diagnosis, prognosis, state of treatment and method of treatment.

49-52. (canceled)
